(19) United States
(12) Patent Application Publication
(10) Pub. No.: US 2024/0124892 A1
(43) Pub. Date: Apr. 18, 2024

(54) ADENO-ASSOCIATED VIRUSES AND METHODS AND MATERIALS FOR MAKING AND USING ADENO-ASSOCIATED VIRUSES

(71) Applicant: UNIVERSITY OF PITTSBURGH-OF THE COMMONWEALTH SYSTEM OF HIGHER EDUCATION, Pittsburgh, PA (US)

(72) Inventors: Leah Byrne, Pittsburgh, PA (US); Bilge Esin Ozturk, Pittsburgh, PA (US)

(21) Appl. No.: 18/277,933

(22) PCT Filed: Mar. 2, 2022
(86) PCT No.: PCT/US2022/018543

§ 371 Date: Aug. 18, 2023

Related U.S. Application Data
(60) Provisional application No. 63/155,624, filed on Mar. 2, 2021.

(51) Int. Cl. C12N 15/86; C07K 14/005

(52) U.S. Cl. CPC C12N 15/86; C07K 14/005; C12N 2750/14122; C12N 2750/14143; C12N 2800/40

(57) ABSTRACT

This document provides AAVs and methods and materials for making and using AAVs. For example, AAVs containing a VP1 polypeptide, a VP2 polypeptide, and a VP3 polypeptide having a heterologous amino acid segment having the ability to bind to a binding partner are provided. This document also provides compositions containing an AAV described herein, a vector system encoding an AAV described herein, and methods for making a composition that includes two or more different AAVs covalently linked together.

### CROSS-REFERENCE TO RELATED APPLICATIONS

This application claims the benefit of U.S. Provisional Application Ser. No. 63/155,624, filed Mar. 2, 2021. The disclosure of the prior application is considered part of (and is incorporated by reference in) the disclosure of this application.

### BACKGROUND

### 1. Technical Field

This document relates to adeno-associated viruses (AAVs) and methods and materials for making and using AAVs. For example, this document provides AAVs containing a modified capsid polypeptide and methods for using such AAVs to produce compositions containing two or more AAVs covalently linked together.

### 2. Background Information

Viruses, such as AAVs, are efficient modes for in vivo gene transfer, and their use in the clinic is expanding. Improved AAV designs and production techniques for making effective AAV preparations should further expand the use of AAVs in the laboratory and clinic.

### SUMMARY

This document provides AAVs and methods and materials for making and using AAVs. For example, this document provides AAVs containing a VP1 polypeptide, a VP2 polypeptide, and a VP3 polypeptide with the VP3 polypeptide containing a heterologous amino acid segment and the VP1 and VP2 polypeptides not containing that heterologous amino acid segment. That heterologous amino acid segment can be any appropriate amino acid segment. In some cases, the heterologous amino acid segment of a VP3 polypeptide can be an amino acid segment having the ability to bind to (e.g., to covalently bind to) a binding partner. For example, heterologous amino acid segment of a VP3 polypeptide can be a spytag sequence, a snooptag sequence, a spycatcher sequence, a snoopcatcher sequence, a DPIVMIDNDKPIT sequence (SEQ ID NO:1) or a variant SEQ ID NO:1. When using a spytag sequence as a heterologous amino acid segment of a VP3 polypeptide as described herein, then the binding partner can be a spycatcher sequence. When using a spycatcher sequence as a heterologous amino acid segment of a VP3 polypeptide as described herein, then the binding partner can be a spytag sequence. Likewise, when using a snooptag sequence as a heterologous amino acid segment of a VP3 polypeptide as described herein, then the binding partner can be a snoopcatcher sequence. When using a snoopcatcher sequence as a heterologous amino acid segment of a VP3 polypeptide as described herein, then the binding partner can be a snooptag sequence.

Having the ability to design AAVs having (a) a VP3 polypeptide containing a heterologous amino acid sequence (e.g., a tag sequence that can bind covalently to a binding partner), and (b) VP1 and VP2 polypeptides that lack that heterologous amino acid sequence as described herein, can allow for the production and use of high amounts of functional AAVs having the ability to bind to other agents such as other AAVs (e.g., AAVs containing a VP3 polypeptide having a binding partner for the heterologous amino acid sequence) or a polypeptide having a binding partner for the heterologous amino acid sequence (e.g., a Cas9 polypeptide containing a binding partner for the heterologous amino acid sequence or a conjugating polypeptide containing a binding partner for the heterologous amino acid sequence). For example, an AAV having a VP3 polypeptide containing a heterologous amino acid sequence (e.g., a tag sequence that can bind covalently to a binding partner) can have the ability to form a covalent bond with a polypeptide designed to include a binding partner that covalently binds to that heterologous amino acid segment of the VP3 polypeptide. For example, a conjugating polypeptide can be designed as described herein to include a binding partner that covalently binds to a heterologous amino acid segment (e.g., a heterologous amino acid segment having SEQ ID NO:1 or a variant thereof) of a VP3 polypeptide of an AAV provided herein and used to conjugate that AAV to, for example, another AAV to form two AAVs that are covalently attached together (see, e.g., FIG. 2).

This document also provides (a) compositions containing an AAV described herein, (b) nucleic acid molecules encoding the components of an AAV described herein, (c) conjugating polypeptides, (d) nucleic acid molecules encoding a conjugating polypeptide described herein, (e) methods for making a composition that includes two or more different AAVs covalently linked together, and (f) methods for making a composition that includes an AAV covalently linked a polypeptide (e.g., a Cas9 polypeptide containing a binding partner for the heterologous amino acid sequence or a conjugating polypeptide containing a binding partner for the heterologous amino acid sequence).

As described herein, AAVs designed to contain (a) VP3 polypeptides that include a heterologous amino acid segment and (b) VP1 and VP2 polypeptides that lack that heterologous amino acid segment can be used to produce high titer preparations of AAVs. For example, AAVs designed to contain (a) VP3 polypeptides that include a heterologous amino acid segment and (b) VP1 and VP2 polypeptides that lack that heterologous amino acid segment can be used to produce a preparation of functional AAVs with a titer greater than 10¹¹ (e.g., greater than 1×10¹¹, greater than 2×10¹¹, greater than 3×10¹¹, greater than 4×10¹¹, greater than 5×10¹¹, greater than 6×10¹¹, greater than 7×10¹¹, greater than 8×10¹¹, or greater than 9×10¹¹, greater than 1×10¹², greater than 2×10¹², greater than 3×10¹², greater than 4×10¹², greater than 5×10¹², greater than 6×10¹², greater than 7×10¹², greater than 8×10¹², or greater than 9×10¹², greater than 1×10¹³, greater than 2×10¹³, greater than 3×10¹³, greater than 4×10¹³, greater than 5×10¹³, greater than 6×10¹³, greater than 7×10¹³, greater than 8×10¹³, or greater than 9×10¹³, greater than 1×10¹⁴, greater than 2×10¹⁴, greater than 3×10¹⁴, greater than 4×10¹⁴, greater than 5×10¹⁴, greater than 6×10¹⁴, greater than 7×10¹⁴, greater than 8×10¹⁴, or greater than 9×10¹⁴). In some cases, an AAV designed to contain (a) VP3 polypeptides that include a heterologous amino acid segment and (b) VP1 and VP2 polypeptides that lack that heterologous amino acid segment can be used to produce an AAV preparation with a titer that is greater than (e.g., at least 5-fold, 10-fold, 25-fold, 50-fold, 75-fold, or 100-fold greater than) an AAV preparation of a comparable AAV that contains the heterologous amino acid segment as part of the VP1, VP2, and VP3 polypeptides.

Having the ability to produce AAV preparations with such high titers of functional AAVs containing a VP3 polypeptide engineered to include a heterologous amino acid segment as described herein can greatly increases the efficiency of using a preparation of AAVs (e.g., modified AAVs) to produce products that include a functional AAV covalently attached to another moiety of interest such as another AAV and/or a polypeptide.

In general, one aspect of this document features an AAV comprising a VP1 polypeptide, a VP2 polypeptide, and a VP3 polypeptide, wherein the VP3 polypeptide comprises a heterologous amino acid segment having the ability to bind to a binding partner, and wherein the VP1 polypeptide and the VP2 polypeptide lack the heterologous amino acid segment. The AAV can be an AAV2. The amino acid segment can comprise a spytag sequence, a snooptag sequence, a spycatcher sequence, a snoopcather sequence, or the amino acid sequence set forth in SEQ ID NO:1. The VP3 polypeptide can comprise the amino acid segment at an amino acid position within three amino acid residues of an amino acid residue corresponding to position 453 or position 588 of a reference VP1 polypeptide having the amino acid sequence set forth in SEQ ID NO:2. The VP3 polypeptide can comprise a protease cleavage site.

In another aspect, this document features a composition comprising (or consisting essentially of or consisting of) an AAV comprising a VP1 polypeptide, a VP2 polypeptide, and a VP3 polypeptide, wherein the VP3 polypeptide comprises a heterologous amino acid segment having the ability to bind to a binding partner, and wherein the VP1 polypeptide and the VP2 polypeptide lack the heterologous amino acid segment. The AAV can be an AAV2. The amino acid segment can comprise a spytag sequence, a snooptag sequence, a spycatcher sequence, a snoopcather sequence, or the amino acid sequence set forth in SEQ ID NO:1. The VP3 polypeptide can comprise the amino acid segment at an amino acid position within three amino acid residues of an amino acid residue corresponding to position 453 or position 588 of a reference VP1 polypeptide having the amino acid sequence set forth in SEQ ID NO:2. The VP3 polypeptide can comprise a protease cleavage site. The titer of the AAV of the composition can be greater than 10¹¹.

In another aspect, this document features an isolated nucleic acid comprising (or consisting essentially of or consisting of) a nucleic acid sequence encoding a VP1 polypeptide, a VP2 polypeptide, and a VP3 polypeptide except that the nucleic acid sequence lacks the start codon of the VP1 polypeptide, and except that the nucleic acid sequence comprises a stop codon that is (a) within the sequence encoding the VP2 polypeptide and upstream of the sequence encoding the VP3 polypeptide. Expression of the isolated nucleic acid sequence can produce the VP3 polypeptide without producing the VP1 polypeptide or the VP2 polypeptide. The VP3 polypeptide can comprise a heterologous amino acid segment having the ability to bind to a binding partner. The amino acid segment can comprise a spytag sequence, a snooptag sequence, a spycatcher sequence, a snoopcather sequence, or the amino acid sequence set forth in SEQ ID NO:1. The VP3 polypeptide can comprise the amino acid segment at an amino acid position within three amino acid residues of an amino acid residue corresponding to position 453 or position 588 of a reference VP1 polypeptide having the amino acid sequence set forth in SEQ ID NO:2. The VP3 polypeptide can comprise a protease cleavage site.

In another aspect, this document features an isolated nucleic acid comprising (or consisting essentially of or consisting of) a nucleic acid sequence encoding a VP1 polypeptide, a VP2 polypeptide, and a VP3 polypeptide except that the nucleic acid sequence lacks the start codon of the VP3 polypeptide. Expression of the isolated nucleic acid sequence can produce the VP1 polypeptide and the VP2 polypeptide without producing the VP3 polypeptide.

In another aspect, this document features a vector system for making an AAV comprising a VP1 polypeptide, a VP2 polypeptide, and a VP3 polypeptide, wherein the VP3 polypeptide comprises a heterologous amino acid segment having the ability to bind to a binding partner, and wherein the VP1 polypeptide and the VP2 polypeptide lack the heterologous amino acid segment, wherein the vector system comprises: (a) a first vector comprising a nucleic acid sequence encoding a VP1 polypeptide, a VP2 polypeptide, and the VP3 polypeptide comprising the heterologous amino acid segment except that the nucleic acid sequence of the first vector lacks the start codon of the VP1 polypeptide, and except that the nucleic acid sequence of the first vector comprises a stop codon that is (a) within the sequence encoding the VP2 polypeptide and upstream of the sequence encoding the VP3 polypeptide, and (b) a second vector comprising a nucleic acid sequence encoding a VP1 polypeptide lacking the heterologous amino acid segment, a VP2 polypeptide lacking the heterologous amino acid segment, and a VP3 polypeptide except that the nucleic acid sequence of the second vector lacks the start codon of the VP3 polypeptide of the second vector. The AAV can be an AAV2. Expression of the first and second vectors can produce the AAV comprising a VP1 polypeptide expressed from the second vector, a VP2 polypeptide expressed from the second vector, and a VP3 polypeptide expressed from the first vector. The heterologous amino acid segment can comprise a spytag sequence, a snooptag sequence, a spycatcher sequence, a snoopcather sequence, or the amino acid sequence set forth in SEQ ID NO:1. The VP3 polypeptide can comprise the heterologous amino acid segment at an amino acid position within three amino acid residues of an amino acid residue corresponding to position 453 or position 588 of a reference VP1 polypeptide having the amino acid sequence set forth in SEQ ID NO:2. The VP3 polypeptide comprising the heterologous amino acid segment can comprise a protease cleavage site.

In another aspect, this document features a composition comprising (or consisting essentially of or consisting of) (a) a first AAV comprising a VP1 polypeptide, a VP2 polypeptide, and a VP3 polypeptide comprising a heterologous amino acid segment having the ability to bind to a binding partner, wherein the VP1 and VP2 polypeptides of the first AAV lack the heterologous amino acid segment, and (b) a second AAV comprising a VP1 polypeptide, a VP2 polypeptide, and a VP3 polypeptide. The AAV can be an AAV2. The heterologous amino acid segment can comprise a spytag sequence, a snooptag sequence, a spycatcher sequence, a snoopcather sequence, or the amino acid sequence set forth in SEQ ID NO:1. The VP3 polypeptide of the first AAV can comprise the heterologous amino acid segment at an amino acid position within three amino acid residues of an amino acid residue corresponding to position 453 or position 588 of a reference VP1 polypeptide having the amino acid sequence set forth in SEQ ID NO:2. The VP3 polypeptide of the first AAV can comprise a protease cleavage site. The VP3 polypeptide of the second AAV can comprise the binding partner. The VP1 and VP2 polypeptides of the second AAV can lack the binding partner. The first and second AAVs can be covalently attached to each other. The first AAV can be covalently attached to the second AAV via at least one covalent bond between the amino acid segment of the VP3 polypeptide of the first AAV and the binding partner of the VP3 polypeptide of the second AAV. The VP3 polypeptide of the second AAV can comprise a heterologous amino acid segment having the ability to bind to a binding partner. The VP1 and VP2 polypeptides of the second AAV can lack the binding partner. The first and second AAVs can be covalently attached to each other. The first AAV can be covalently attached to the second AAV via a conjugating polypeptide comprising (a) the binding partner of the heterologous amino acid segment of the VP3 polypeptide of the first AAV and (b) the binding partner of the heterologous amino acid segment of the VP3 polypeptide of the second AAV.

In another aspect, this document features a method for producing a composition comprising (or consisting essentially of or consisting of) a first AAV covalently linked to a second AAV, wherein the first AAV comprises a VP1 polypeptide, a VP2 polypeptide, and a VP3 polypeptide comprising a heterologous amino acid segment having the ability to bind to a binding partner, wherein the VP1 and VP2 polypeptides of the first AAV lack the heterologous amino acid segment, and wherein the second AAV comprises a VP1 polypeptide, a VP2 polypeptide, and a VP3 polypeptide comprising the binding partner, wherein the method comprises contacting the first AAV with the second AAV to form a first AAV-second AAV complex, thereby producing the composition. The first AAV can be an AAV2. The second AAV can be an AAV2. The heterologous amino acid segment can comprise a spytag sequence, a snooptag sequence, a spycatcher sequence, a snoopcather sequence, or the amino acid sequence set forth in SEQ ID NO:1. The VP3 polypeptide of the first AAV can comprise the heterologous amino acid segment at an amino acid position within three amino acid residues of an amino acid residue corresponding to position 453 or position 588 of a reference VP1 polypeptide having the amino acid sequence set forth in SEQ ID NO:2. The VP3 polypeptide of the first AAV can comprise a protease cleavage site. The VP1 and VP2 polypeptides of the second AAV can lack the binding partner. The first and second AAVs of the first AAV-second AAV complex can be covalently attached to each other. The first AAV can be covalently attached to the second AAV via at least one covalent bond between the amino acid segment of the VP3 polypeptide of the first AAV and the binding partner of the VP3 polypeptide of the second AAV.

In another aspect, this document features a method for producing a composition comprising (or consisting essentially of or consisting of) a first AAV attached to a second AAV, wherein the first AAV comprises a VP1 polypeptide, a VP2 polypeptide, and a VP3 polypeptide comprising a heterologous amino acid segment having the ability to bind to a binding partner, wherein the VP1 and VP2 polypeptides of the first AAV lack the heterologous amino acid segment, and wherein the second AAV comprises a VP1 polypeptide, a VP2 polypeptide, and a VP3 polypeptide comprising the binding partner, wherein the method comprises contacting the first AAV with the second AAV to form a first AAV-second AAV complex, thereby producing the composition. The first AAV can be an AAV2. The second AAV can be an AAV2. The heterologous amino acid segment can comprise a spytag sequence, a snooptag sequence, a spycatcher sequence, a snoopcather sequence, or the amino acid sequence set forth in SEQ ID NO:1. The VP3 polypeptide of the first AAV can comprise the heterologous amino acid segment at an amino acid position within three amino acid residues of an amino acid residue corresponding to position 453 or position 588 of a reference VP1 polypeptide having the amino acid sequence set forth in SEQ ID NO:2. The VP3 polypeptide of the first AAV can comprise a protease cleavage site. The VP1 and VP2 polypeptides of the second AAV can lack the binding partner. The first and second AAVs of the first AAV-second AAV complex can be covalently attached to each other. The first AAV can be covalently attached to the second AAV via at least one covalent bond between the amino acid segment of the VP3 polypeptide of the first AAV and the binding partner of the VP3 polypeptide of the second AAV.

In another aspect, this document features a method for producing a composition comprising (or consisting essentially of or consisting of) a first AAV attached to a second AAV, wherein the first AAV comprises a VP1 polypeptide, a VP2 polypeptide, and a VP3 polypeptide comprising a first heterologous amino acid segment having the ability to bind to a first binding partner, wherein the VP1 and VP2 polypeptides of the first AAV lack the first heterologous amino acid segment, and wherein the second AAV comprises a VP1 polypeptide, a VP2 polypeptide, and a VP3 polypeptide comprising a second heterologous amino acid segment having the ability to bind to a second binding partner, wherein the method comprises (a) contacting the first AAV with a conjugating polypeptide to form a first AAV-conjugating polypeptide complex, wherein the conjugating polypeptide comprises the first binding partner and the second binding partner, and (b) contacting the first AAV-conjugating polypeptide complex with the second AAV to form a first AAV-conjugating polypeptide-second AAV complex, thereby producing the composition comprising the first AAV attached to the second AAV. The first AAV can be an AAV2. The second AAV can be an AAV2. The first heterologous amino acid segment can comprise a spytag sequence, a snooptag sequence, a spycatcher sequence, a snoopcather sequence, or the amino acid sequence set forth in SEQ ID NO:1. The second heterologous amino acid segment can comprise a spytag sequence, a snooptag sequence, a spycatcher sequence, a snoopcather sequence, or the amino acid sequence set forth in SEQ ID NO:1. The VP3 polypeptide of the first AAV can comprise the first heterologous amino acid segment at an amino acid position within three amino acid residues of an amino acid residue corresponding to position 453 or position 588 of a reference VP1 polypeptide having the amino acid sequence set forth in SEQ ID NO:2. The VP3 polypeptide of the second AAV can comprise the second heterologous amino acid segment at an amino acid position within three amino acid residues of an amino acid residue corresponding to position 453 or position 588 of a reference VP1 polypeptide having the amino acid sequence set forth in SEQ ID NO:2. The VP3 polypeptide of the first AAV or the second AAV can comprise a protease cleavage site. The VP1 and VP2 polypeptides of the second AAV can lack the second heterologous amino acid segment. The first and second AAVs of the first AAV-conjugating polypeptide-second AAV complex can be covalently attached to each other. The first AAV can be covalently attached to the conjugating polypeptide via at least one covalent bond between the first heterologous amino acid segment and the first binding partner of the conjugating polypeptide, and wherein the second AAV can be covalently attached to the conjugating polypeptide via at least one covalent bond between the second heterologous amino acid segment and the second binding partner of the conjugating polypeptide. The contacting step with the first AAV or the second AAV can comprise using a solution comprising a titer of the first AAV or of the second AAV that is greater than 10¹¹.

In another aspect, this document features a method for producing a composition comprising (or consisting essentially of or consisting of) a first AAV attached to a second AAV, wherein the first AAV comprises a VP1 polypeptide, a VP2 polypeptide, and a VP3 polypeptide comprising a first heterologous amino acid segment having the ability to bind to a first binding partner, wherein the VP1 and VP2 polypeptides of the first AAV lack the first heterologous amino acid segment, and wherein the second AAV comprises a VP1 polypeptide, a VP2 polypeptide, and a VP3 polypeptide comprising a second heterologous amino acid segment having the ability to bind to a second binding partner, wherein the method comprises (a) contacting the second AAV with a conjugating polypeptide to form a second AAV-conjugating polypeptide complex, wherein the conjugating polypeptide comprises the first binding partner and the second binding partner, and (b) contacting the second AAV-conjugating polypeptide complex with the first AAV to form a first AAV-conjugating polypeptide-second AAV complex, thereby producing the composition comprising the first AAV attached to the second AAV. The first AAV can be an AAV2. The second AAV can be an AAV2. The first heterologous amino acid segment can comprise a spytag sequence, a snooptag sequence, a spycatcher sequence, a snoopcather sequence, or the amino acid sequence set forth in SEQ ID NO:1. The second heterologous amino acid segment can comprise a spytag sequence, a snooptag sequence, a spycatcher sequence, a snoopcather sequence, or the amino acid sequence set forth in SEQ ID NO:1. The VP3 polypeptide of the first AAV can comprise the first heterologous amino acid segment at an amino acid position within three amino acid residues of an amino acid residue corresponding to position 453 or position 588 of a reference VP1 polypeptide having the amino acid sequence set forth in SEQ ID NO:2. The VP3 polypeptide of the second AAV can comprise the second heterologous amino acid segment at an amino acid position within three amino acid residues of an amino acid residue corresponding to position 453 or position 588 of a reference VP1 polypeptide having the amino acid sequence set forth in SEQ ID NO:2. The VP3 polypeptide of the first AAV or the second AAV can comprise a protease cleavage site. The VP1 and VP2 polypeptides of the second AAV can lack the second heterologous amino acid segment. The first and second AAVs of the first AAV-conjugating polypeptide-second AAV complex can be covalently attached to each other. The first AAV can be covalently attached to the conjugating polypeptide via at least one covalent bond between the first heterologous amino acid segment and the first binding partner of the conjugating polypeptide, and wherein the second AAV can be covalently attached to the conjugating polypeptide via at least one covalent bond between the second heterologous amino acid segment and the second binding partner of the conjugating polypeptide. The contacting step with the first AAV or the second AAV can comprise using a solution comprising a titer of the first AAV or of the second AAV that is greater than 10¹¹.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a schematic of two AAVs covalently attached together according to one embodiment.

FIG. 2 is a schematic of two AAVs covalently attached together using a conjugating polypeptide according to one embodiment.

FIG. 3 is a schematic of a vector system that can be used to produce AAVs having a VP1 polypeptide, a VP2 polypeptide, and a VP3 polypeptide with the VP3 polypeptide containing a heterologous amino acid segment (expressed from middle AAV trans vector) and the VP1 and VP2 polypeptides not containing that heterologous amino acid segment (expressed from the far right AAV trans vector). The AAV cis vector (far left) can be designed to express any gene of interest (e.g., a nucleic acid encoding a polypeptide of interest such as a therapeutic polypeptide, a fluorescent polypeptide, and/or a gene editing component).

FIG. 4 is a table of an exemplary transfection protocol for producing AAV according to one embodiment.

FIG. 5 contains images of green fluorescent protein (GFP) in HEK 293 cells following infection with (a) AAV2 containing wild-type VP1, VP2, and VP3 polypeptides expressed from a complete AAV2 vector (AAV2), (b) AAV2-588-TG-SpyTag-GLS, which contains VP1, VP2, and VP3 polypeptides with each containing the TG linker followed by a spytag sequence followed by a GLS linker inserted at position 588, (c) AAV2 that contains a wild-type VP3 polypeptide and no VP1 polypeptides and no VP2 polypeptides (VP1/2 Stop), (d) AAV2 that contains wild-type VP1 and VP2 polypeptides and no VP3 polypeptides (VP1/2 (VP3 STOP) (Isoleucine mutation)), and (e) AAV2 that contains wild-type VP1, VP2, and VP3 polypeptides with the VP1 and VP2 polypeptide being produced from a vector that lacks the VP3 start codon and with the VP3 polypeptide being produced from a vector that lacks the VP1 start codon and contains a stop codon within the VP2 coding sequence upstream of the VP3 coding sequence.

FIG. 6 is a table of the AAV titers produced using the indicated combination of AAV vectors.

FIG. 7 is an immunoblot showing AAV-453-FlagTag-SpyTag vector linked to Spycatcher/SnoopCatcher conjugating polypeptide with staining using a mouse monoclonal antibody against VP1, VP2, and VP3 from Progen and a rabbit monoclonal antibody against the Flag tag sequence from Abcam.

FIG. 8 is an immunoblot showing AAV-588-LFL-SpyTag vector linked to both Spycatcher/SnoopCatcher and Spycatcher-Flag-SnoopCatcher conjugating polypeptides with staining using a mouse monoclonal antibody against VP1, VP2, and VP3 from Progen and a rabbit monoclonal antibody against the Flag tag sequence from Abcam.

FIG. 9 is a graph quantitatively plotting fluorescence vs. molecular weight for the immunoblot results from FIG. 8.

FIG. 10 is an immunoblot showing the linking between Cas9-SnoopTag fusion polypeptide with different amounts of Spycatcher/SnoopCatcher conjugating polypeptides.

FIG. 11 is a photograph of an agarose gel showing the results of a T7 endonuclease assay, demonstrating the editing efficiency of Cas9-Snooptag.

FIG. 12 is a sequence listing of the amino acid sequence (SEQ ID NO:2) of a reference VP1 capsid polypeptide of AAV2. *=position 588 for possible insertion.

FIG. 13 is a schematic of a second AAV (70) including a VP3 polypeptide containing a binding partner (72). Protease cleavage (60) of a first AAV (50) releases one end of the amino acid segment (54), and a covalently bond between the amino acid segment and the binding partner binds the first AAV and the second AAV together.

FIG. 14 is a sequence listing of nucleic acid and amino acid sequences of VP1/2 (VP3 Start Codon Methionine to Serine Mutation).

FIG. 15 is a sequence listing of nucleic acid and amino acid sequences of VP1/2 (VP3 Start Codon Methionine to Leucine Mutation).

FIG. 16 is a sequence listing of nucleic acid and amino acid sequences of VP1/2 (VP3 Start Codon Methionine to Isoleucine Mutation).

FIG. 17 is a sequence listing of nucleic acid and amino acid sequences of VP3-453-FlagTag-SnoopTag (VP1/2 STOP).

FIG. 18 is a sequence listing of nucleic acid and amino acid sequences of VP3-588-Long Flexible Linker-SPYTAG (VP1/2 STOP).

FIG. 19 is a sequence listing of nucleic acid and amino acid sequences of VP3-453-SPYTAG-SPYTAG (VP1/2 STOP).

FIG. 20 is a sequence listing of nucleic acid and amino acid sequences of VP3-453-SNOOPTAG-SNOOPTAG (VP1/2 STOP).

FIG. 21 is a sequence listing of nucleic acid and amino acid sequences of VP3-453-HisTag-TEV-SpyTag (VP1/2 STOP).

FIG. 22 is a sequence listing of nucleic acid and amino acid sequences of VP3-453-SpyTag002 X2 (VP1/2 STOP).

FIG. 23 is a sequence listing of nucleic acid and amino acid sequences of VP3-588-SnoopTag X2 (VP1/2 STOP).

FIG. 24 is a sequence listing of nucleic acid and amino acid sequences of VP3-453-LFL-SpyTag (VP1/2 STOP).

FIG. 25 is a sequence listing of nucleic acid and amino acid sequences of VP3-453-FlagTag-SpyTag (VP1/2 STOP).

FIG. 26 is a sequence listing of nucleic acid and amino acid sequences of VP3-588-LFL-SnoopTag (VP1/2 STOP).

FIG. 27 is a sequence listing of nucleic acid and amino acid sequences of FLEX, which is a SPYCATCHER-LONG FLEXIBLE LINKER-SNOOPCATCHER conjugating polypeptide.

FIG. 28 is a sequence listing of nucleic acid and amino acid sequences of a FLAGTAG-SPYCATCHER-SNOOPCATCHER conjugating polypeptide.

FIG. 29 is a sequence listing of nucleic acid and amino acid sequences of a SPYCATCHER-6XHIS-FLAGTAG-SNOOPCATCHER conjugating polypeptide.

FIG. 30 is a sequence listing of nucleic acid and amino acid sequences of a spCAS9-SNOOPTAG polypeptide.

### DETAILED DESCRIPTION

This document provides AAVs containing (a) a VP3 polypeptide containing a heterologous amino acid segment (e.g., a tag sequence that can bind covalently to a binding partner) and (b) VP1 and VP2 polypeptides that lack that heterologous amino acid segment. An appropriate AAV can be engineered to contain (a) a VP3 polypeptide containing a heterologous amino acid segment (e.g., a tag sequence that can bind covalently to a binding partner) and (b) VP1 and VP2 polypeptides that lack that heterologous amino acid segment. For example, AAV1, AAV2, AAV4, AAV5, AAV6, AAV7, AAV8, and AAV9 can be engineered to contain (a) a VP3 polypeptide containing a heterologous amino acid segment (e.g., a tag sequence that can bind covalently to a binding partner) and (b) VP1 and VP2 polypeptides that lack that heterologous amino acid segment. In some cases, an AAV2 can be engineered to include (a) a VP3 polypeptide containing a heterologous amino acid segment (e.g., a tag sequence that can bind covalently to a binding partner) and (b) VP1 and VP2 polypeptides that lack that heterologous amino acid segment.

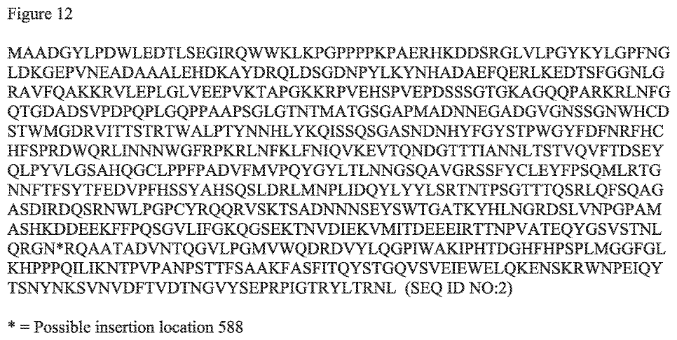

As described herein, an AAV (e.g., AAV2) can be designed such that a heterologous amino acid segment is inserted into a VP3 polypeptide of that AAV without be present within the VP1 and VP2 polypeptides of that AAV. Any appropriate site of a VP3 polypeptide can be used as the site for accommodating insertion of a heterologous amino acid segment (e.g., a heterologous amino acid segment having the ability to bind to a binding partner). Examples of such sites include, without limitation, those amino acid positions of a VP3 polypeptide that correspond to amino acid position 262, amino acid position 453, amino acid position 587, amino acid position 588, and amino acid position 589 of a reference VP1 polypeptide with the amino acid sequence set forth in SEQ ID NO:2 (FIG. 12). The numbering of VP1 is used with respect to VP1, VP2, and VP3 as VP2 and VP3 are polypeptides that use alternative start sites within VP1. In one example, an AAV2 can be engineered to include a VP3 polypeptide that includes a heterologous amino acid segment (e.g., a tag sequence having the ability to covalently bind to a binding partner) located at position 588 of a VP3 polypeptide (which is based on the VP1 amino acid numbering) of that AAV2.

As described herein, any appropriate heterologous amino acid segment can be inserted into a VP3 polypeptide. For example, a heterologous amino acid segment having the ability to bind to (e.g., to covalently bind to) a binding partner can be inserted into a VP3 polypeptide. Examples of heterologous amino acid segments having the ability to bind to (e.g., to covalently bind to) a binding partner and that can be used as described herein include, without limitation, a spytag sequence that can bind to a spycatcher sequence, a spycatcher sequence that can bind to a spytag sequence, a snooptag sequence that can bind to a snoopcatcher sequence, a snoopcatcher sequence that can bind to a snooptag sequence, the amino acid sequence set forth in SEQ ID NO:1 (or a variant of the amino acid sequence set forth in SEQ ID NO:1) that can bind to a sequence described herein (e.g., SEQ ID NO:56), and a sequence described herein (e.g., SEQ ID NO:56) that can bind to the amino acid sequence set forth in SEQ ID NO:1 (or a variant of the amino acid sequence set forth in SEQ ID NO:1). Additional examples heterologous amino acid segments and binding partner pairs that can be used as described herein are set forth in Table 1.

TABLE 1
Amino acid segment and corresponding
binding partner (or vice versa).

| | | SEQ | | SEQ |
|---|---|---|---|---|
| | | ID | | ID |
| Example | Segment | NO: | Binding Partner | NO: |
| 1 | AHIVMVD | 69 | DSATHIKFSKRDEDGKELAG | 70 |
| | AYKPTK | | ATMELRDSSGKTISTWISDGQ | |
| | | | VKDFYLYPGKYTFVETAAPD | |
| | | | GYEVATAITFTVNEQGQVTV | |
| | | | NGKATKGDAHI | |
| 2 | KLGDIEF | 71 | GSHMKPLRGAVFSLQKQHPD | 72 |
| | IKVNK | | YPDIYGAIDQNGTYQNVRTG | |
| | | | EDGKLTFKNLSDGKYRLFEN | |
| | | | SEPAGYKPVQNKPIVAFQIV | |
| | | | NGEVRDVTSIVPQDIPATYE | |
| | | | FTNGKHYITNEPIPPK | |
| 3 | VPTIVMV | 73 | AMVTTLSGLSGEQGPSGDMT | 74 |
| | DAYKRYK | | TEEDSATHIKFSKRDEDGRE | |
| | | | LAGATMELRDSSGKTISTWI | |
| | | | SDGHVKDFYLYPGKYTFVET | |
| | | | AAPDGYEVATAITFTVNEQG | |
| | | | QVTVNGEATKGDAHTGSSGS | |

Any appropriate variant of the amino acid sequence set forth in SEQ ID NO:1 can be used. For example, a variant of the amino acid sequence set forth in SEQ ID NO:1 can be designed to include one, two, three, four, five, six, or seven amino acid additions, deletions, or substitutions and can be used as described herein. In some cases, a variant of the amino acid sequence set forth in SEQ ID NO:1 can be designed to include one, two, or three amino acid additions, deletions, or substitutions. Examples of variants of the amino acid sequence set forth in SEQ ID NO:1 that can be used as described herein include, without limitation, those set forth in Table 2.

TABLE 2
Examples of variant of SEQ ID NO: 1.

| Variant | | SEQ ID |
|---|---|---|
| Number | Sequence | NO: |
| 1 | APIVMIDNDKPIT | 40 |
| 2 | DHIVMIDNDKPIT | 41 |
| 3 | DPIVMVDNDKPIT | 42 |
| 4 | DPIVMIDADKPIT | 43 |
| 5 | DPIVMIDNYKPIT | 44 |
| 6 | DPIVMIDNDKPYT | 45 |
| 7 | DPIVMIDNDKPIT | 46 |
| 8 | GSDPIVMIDNDKPITGS | 47 |
| 9 | PIVMIDNDKPIT | 48 |
| 10 | PIVMIDNDKPI | 49 |

A binding partner having the ability to form a covalent bond with an amino acid segment containing the amino acid sequence set forth in SEQ ID NO:1 (or a variant thereof) that can be used as described herein can have any appropriate amino acid sequence. Examples of binding partner sequences having the ability to form a covalent bond with an amino acid segment containing the amino acid sequence set forth in SEQ ID NO:1 (or a variant thereof) that can be used as described herein include, without limitation, those set forth in the following formula:

DSTTH X₁ KFSKRDING X₂ ELAGA X₃ IELRN X₄ SG
X₅ TIQSW X₆ SDG X₇ VKDFYLMPG X₈ YQFVETAAPE
GYEL X₉ APITFTIDE X₁₀ GQIWV X₁₁ S

where X₁ to X₁₁=any of the twenty amino acids (SEQ ID NO:56).

Other examples of binding partner sequences having the ability to form a covalent bond with an amino acid segment containing the amino acid sequence set forth in SEQ ID NO:1 (or a variant thereof) that can be used as described herein include, without limitation, those set forth in the following formula, designated herein as SEQ ID NO:57:

DSTTH X₁ KFSKRDING X₂ ELAGA X₃
IELRN X₄ SG X₅ TIQSW X₆ SDG X₇
VK(D/V)FYLMPG X₈ YQFVETAAPEGY
EL X₉ APITFTIDE X₁₀ GQIWV X₁₁ S

where:
X₁=valine, alanine, isoleucine, or leucine;
X₂=lysine, arginine, histidine, or serine;
X₃=methionine, leucine, phenylalanine, or tryptophan
X₄=leucine, methionine, isoleucine, or tyrosine;
X₅=glutamine, serine, threonine, or asparagine;
X₆=isoleucine, leucine, methionine, or phenylalanine;
X₇=threonine, serine, valine, or asparagine;
X₈=threonine, serine, valine, or asparagine;
X₉=alanine, isoleucine, leucine, or phenylalanine;
X₁₀=lysine, arginine, histidine, or serine; and
X₁₁=aspartic acid, glutamic acid, or lysine.

Other examples of binding partner sequences having the ability to form a covalent bond with an amino acid segment that contains the amino acid sequence set forth in SEQ ID NO:1 (or a variant thereof) that can be used as described herein include, without limitation, those set forth in the following formula:

IDT X₁ SGLS X₂ X₃ T X₄ X₅ S X₆ NTT X₇
EEDSTTH X₈ KFSKRDING X₉ ELAGA X₁₀
IELRN X₁₁ SG X₁₂ TIQSW X₁₃ SDG X₁₄ VK
(D/V)FYLMPG X₁₅ YQFVETAAPEGYEL X₁₆
APITFTIDE X₁₇ GQIWV X₁₈ S X₁₉ LIVGDDP
X₂₀ VMIDNDKPIT

where X₁ to X₂₀=any of the twenty amino acids (SEQ ID NO:58).

Still other examples of binding partner sequences having the ability to form a covalent bond with an amino acid segment containing the amino acid sequence set forth in SEQ ID NO:1 (or a variant thereof) that can be used as described herein include, without limitation, those set forth in the following formula, designated herein as SEQ ID NO:59:

IDT X₁ SGLS X₂ X₃ T X₄ X₅ S X₆ NTT
X₇ EEDSTTH X₈ KFSKRDING X₉ ELAGA
X₁₀ IELRN X₁₁ SG X₁₂ TIQSW X₁₃ SDG
X₁₄ VK(D/V)FYLMPG X₁₅ YQFVETAAPE
GYEL X₁₆ APITFTIDE X₁₇ GQIWV X₁₈
S X₁₉ LIVGDDP X₂₀ VMIDNDKPIT

where
X₁=methionine, isoleucine, leucine, or phenylalanine;
X₂=glycine, proline, cysteine, or serine;
X₃=glutamic acid, aspartic acid, arginine, or lysine;
X₄=glycine, proline, cysteine, or serine;
X₅=glutamine, serine, threonine, or asparagine;
X₆=glycine, proline, cysteine, or serine;
X₇=isoleucine, leucine, methionine, or phenylalanine;
X₈=valine, alanine, isoleucine, or leucine;
X₉=lysine, arginine, histidine, or serine;
X₁₀=methionine, leucine, phenylalanine, or tryptophan;
X₁₁=leucine, methionine, isoleucine, or tyrosine;
X₁₂=glutamine, serine, threonine, or asparagine;
X₁₃=isoleucine, leucine, methionine, or phenylalanine;
X₁₄=threonine, serine, valine, or asparagine;
X₁₅=threonine, serine, valine, or asparagine;
X₁₆=alanine, isoleucine, leucine, or phenylalanine;
X₁₇=lysine, arginine, histidine, or serine;
X₁₈=aspartic acid, glutamic acid, or lysine;
X₁₉=threonine, serine, valine, or asparagine;
X₂₀=isoleucine, leucine, methionine, or phenylalanine.

In some cases, a binding partner sequence having the ability to form a covalent bond with an amino acid segment containing the amino acid sequence set forth in SEQ ID NO:1 (or a variant thereof) that can be used as described herein can have the amino acid sequence as set forth in any one of the sequences of Table 3 (any one of SEQ ID NOs:60-65). In some cases, a binding partner sequence having the ability to form a covalent bond with an amino acid segment containing the amino acid sequence set forth in SEQ ID NO:1 (or a variant thereof) that can be used as described herein can have the amino acid sequence as set forth any one of SEQ ID NOs:60-65 with 20 or less (e.g., one, two, three, four, five, six, seven, eight, nine, ten, 11, 12, 13, 14, 15, 16, 17, 18, 19, or 20) amino acid additions, deletions, or substitutions with the proviso that the underlined amino acid residue remains unchanged. For example, a binding partner sequence having the ability to form a covalent bond with an amino acid segment containing the amino acid sequence set forth in SEQ ID NO:1 (or a variant thereof) that can be used as described herein can have the amino acid sequence as set forth in SEQ ID NO:60 with one, two, three, four, or five amino acid additions, deletions, or substitutions with the proviso that the amino acid located at a position corresponding to position 10 of SEQ ID NO:60 remains a lysine and with the proviso that the amino acid located at a position corresponding to position 56 of SEQ ID NO:60 remains a glutamic acid.

TABLE 3
Examples of binding partner sequences
having the ability to form a covalent
bond with SEQ ID NO: 1 (or a variant
thereof).

| | SEQ |
|---|---|
| | ID |
| Sequence | NO: |
| DSTTHVKFSKRDINGKELAGAMIELRNLSGQ | 60 |
| TIQSWVSDGTVK(D/V)FYLMPGTYQFVETA | |
| APEGYELAAPITFTIDEKGQIWVDS | |
| IDTMSGLSGETGQSGNTTIEEDSTTHVKFSKR | 61 |
| DINGKELAGAMIELRNLSGQTIQSWVSDGTVK | |
| (D/V)FYLMPGTYQFVETAAPEGYELAAPITF | |
| TIDEKGQIWVDSTLIVGDDPIVMIDNDKPIT | |
| DTMSGLSGETGQSGNTTIEEDSTTHVKFSKRD | 62 |
| INGKELAGAMIELRNLSGQTIQSWVSDGTVK | |
| (D/V)FYLMPGTYQFVETAAPEGYELAAPIT | |
| FTIDEKGQIWVDSTLIVGDDP | |
| DTMSGLSGETGQSGNTTIEEDSTTHVKFSKRD | 63 |
| INGKELAGAMIELRNLSGQTIQSWVSDGTVK | |
| (D/V)FYLMPGTYQFVETAAPEGYELAAPI | |
| TFTIDEKGQIWVDS | |
| DSTTHVKFSKRDINGKELAGAMIELRNLSGQT | 64 |
| IQSWVSDGTVK(D/V)FYLMPGTYQFVETAAP | |
| EGYELAAPITFTIDEKGQIWVDSTLIVGDDPI | |
| VMIDNDKPIT | |
| DTMSGLSGETGQSGNTTIEEDSTTHVKFSKRD | 65 |
| INGKELAGAMIELRNLSGQTIQSWVSDGTVK | |
| (D/V)FYLMPGTYQFVETAAPEGYELAAPIT | |
| FTIDEKGQIWVDSTLIVGDDPIVMIDNDKPIT | |

In some cases, an AAV described herein can be designed to include one or more linkers within a VP3 polypeptide. For example, an AAV described herein can be designed to include (a) a linker located N-terminal to the heterologous amino acid segment, (b) a linker located C-terminal to the heterologous amino acid segment, or (c) a linker located N-terminal and a linker located C-terminal to the heterologous amino acid segment. Such linkers can be immediately adjacent to the heterologous amino acid segment or can be within 10, 15, 20, 25, or 30 amino acid residues of the heterologous amino acid segment. For example, an AAV described herein can be designed to include, within a VP3 polypeptide in an N-terminal to C-terminal direction, a VP3 amino acid sequence immediately followed by a first linker immediately followed by the heterologous amino acid segment (e.g., a heterologous amino acid segment having the ability to bind to a binding partner) immediately followed by a second linker immediately followed a continuation of the VP3 amino acid sequence. In another example, an AAV described herein can be designed to include, within a VP3 polypeptide in an N-terminal to C-terminal direction, a VP3 amino acid sequence immediately followed by a first linker followed within 10 amino acid residues by the heterologous amino acid segment (e.g., a heterologous amino acid segment having the ability to bind to a binding partner) followed within 10 amino acid residues by a second linker immediately followed a continuation of the VP3 amino acid sequence. In another example, an AAV described herein can be designed to include, within a VP3 polypeptide in an N-terminal to C-terminal direction, a VP3 amino acid sequence immediately followed by a first linker immediately followed by a continuation of the VP3 amino acid sequence that is less than 20 amino acid residues in length immediately followed by the heterologous amino acid segment (e.g., a heterologous amino acid segment having the ability to bind to a binding partner) immediately followed a continuation of the VP3 amino acid sequence that is less than 20 amino acid residues in length immediately followed by a second linker immediately followed a continuation of the VP3 amino acid sequence.

Any appropriate amino acid or amino acid sequence can be used as a linker to flank (e.g., immediately flank or flank within 10, 15, 20, 25, or 30 amino acid residues) one or both ends of a heterologous amino acid segment (e.g., a heterologous amino acid segment having the ability to bind to a binding partner) that is inserted into a VP3 polypeptide. In some cases, a single amino acid can be used as a linker. Examples of linkers that can be used to flank (e.g., immediately flank or flank within 10, 15, 20, 25, or 30 amino acid residues) one or both ends of a heterologous amino acid segment (e.g., a heterologous amino acid segment having the ability to bind to a binding partner) include, without limitation, leucine-alanine (LA), alanine (A), threonine-glycine (TG), glycine-leucine-serine (GLS), glycine-glycine-serine (GGS), and glycine-serine (GS).

When an AAV provided herein is designed to include two or more linkers within a VP3 polypeptide, those linkers can be the same or different. For example, an AAV provided herein can be designed to include a VP3 polypeptide that contains (a) a GLS linker N-terminal to (e.g., immediately N-terminal to or within 10, 15, 20, 25, or 30 amino acid residues to) a heterologous amino acid segment (e.g., a heterologous amino acid segment having the ability to bind to a binding partner) and (b) a GLS linker C-terminal to (e.g., immediately C-terminal to or within 10, 15, 20, 25, or 30 amino acid residues to) the heterologous amino acid segment. In another example, an AAV provided herein can be designed to include a VP3 polypeptide that contains (a) an LA linker N-terminal to (e.g., immediately N-terminal to or within 10, 15, 20, 25, or 30 amino acid residues to) a heterologous amino acid segment (e.g., a heterologous amino acid segment having the ability to bind to a binding partner) and (b) an A linker C-terminal to (e.g., immediately C-terminal to or within 10, 15, 20, 25, or 30 amino acid residues to) the heterologous amino acid segment. In another example, an AAV provided herein can be designed to include a VP3 polypeptide that contains (a) an TG linker N-terminal to (e.g., immediately N-terminal to or within 10, 15, 20, 25, or 30 amino acid residues to) a heterologous amino acid segment (e.g., a heterologous amino acid segment having the ability to bind to a binding partner) and (b) an GLS linker C-terminal to (e.g., immediately C-terminal to or within 10, 15, 20, 25, or 30 amino acid residues to) the heterologous amino acid segment.

In some cases, an AAV described herein can be designed to include one or more (e.g., one, two, three, four, or five) protease cleavage sites within a VP3 polypeptide. For example, an AAV described herein can be designed to include (a) one or more protease cleavage sites located within a VP3 polypeptide N-terminal to a heterologous amino acid segment (e.g., a heterologous amino acid segment having the ability to bind to a binding partner), or (b) one or more protease cleavage sites located within a VP3 polypeptide C-terminal to a heterologous amino acid segment (e.g., a heterologous amino acid segment having the ability to bind to a binding partner). Typically, the one or more protease cleavage sites are designed to be located within a VP3 polypeptide either N-terminal to the heterologous amino acid segment (e.g., a heterologous amino acid segment having the ability to bind to a binding partner) or C-terminal to the heterologous amino acid segment, but not both. For example, when an AAV described herein is designed to include one or more protease cleavage sites located within a VP3 polypeptide N-terminal to a heterologous amino acid segment (e.g., a heterologous amino acid segment having the ability to bind to a binding partner), the VP3 polypeptide can lack those protease cleavage sites C-terminal to the heterologous amino acid segment. Likewise, when an AAV described herein is designed to include one or more protease cleavage sites located within a VP3 polypeptide C-terminal to a heterologous amino acid segment (e.g., a heterologous amino acid segment having the ability to bind to a binding partner), the VP3 polypeptide can lack those protease cleavage sites N-terminal to the heterologous amino acid segment.

In some cases, one or more protease cleavage sites can be engineered into a VP3 polypeptide described herein such that the site(s) are located immediately adjacent to (either N-terminal to or C-terminal to) a heterologous amino acid segment (e.g., a heterologous amino acid segment having the ability to bind to a binding partner) or can be within 10, 15, 20, 25, or 30 amino acid residues of (either N-terminal to or C-terminal to) a heterologous amino acid segment (e.g., a heterologous amino acid segment having the ability to bind to a binding partner). For example, an AAV described herein can be designed to include, within a VP3 polypeptide in an N-terminal to C-terminal direction, a VP3 amino acid sequence immediately followed by one or more protease cleavage sites immediately followed by a heterologous amino acid segment (e.g., a heterologous amino acid segment having the ability to bind to a binding partner) immediately followed by a continuation of the VP3 amino acid sequence. In another example, an AAV described herein can be designed to include, within a VP3 polypeptide in an N-terminal to C-terminal direction, a VP3 amino acid sequence immediately followed by a heterologous amino acid segment (e.g., a heterologous amino acid segment having the ability to bind to a binding partner) immediately followed by one or more protease cleavage sites immediately followed by a continuation of the VP3 amino acid sequence.

Any appropriate protease cleavage site can be used to create an AAV described herein. For example, the following protease cleavage site of a TEV protease can be used as described herein: an ENLYFQ↓G sequence (SEQ ID NO:50). Other examples of protease cleavage sites (with their corresponding protease) include, without limitation, those set forth in Table 4.

TABLE 4
Examples of protease cleavage sites.

| | Protease Cleavage | SEQ ID |
|---|---|---|
| Protease | Site Sequence | NO: |
| PreScission | LEVLF(Q/G)P | 51 |
| Thrombin | LVPRGS | 52 |
| Factor Xa | I(E/P)GR | 53 |
| Enterokinase | DDDDK | 54 |
| CleanCut | LVPWELQ↓E | 55 |

In some cases, an AAV described herein can be designed to include one or more (e.g., one, two, three, four, or five) protease cleavage sites in combination with one or more linkers within a VP3 polypeptide. Examples of combinations of protease cleavage sites and linkers that can be used to make an AAV described herein include, without limitation, those set forth in Table 5. Each dash located between two listed components in Table 5 can represent that those two components are either immediately contiguous or within 1, 2, 3, 4, 5, 6, 7, 8, 9, 10, 15, 20, 25, or 30 amino acid residues of each other. In some cases, when locate within 1, 2, 3, 4, 5, 6, 7, 8, 9, 10, 15, 20, 25, or 30 amino acid residues of each other, the intervening amino acid(s) can be of the VP3 polypeptide.

TABLE 5
Examples of combinations of protease
cleavage sites and linkers.

| | Order (N-terminal to |
|---|---|
| Example | C-terminal direction) |
| 1 | L-PCS-AAS |
| 2 | L-PCS-L-AAS |
| 3 | L-PCS-AAS-L |
| 4 | L-PCS-L-AAS-L |
| 5 | PCS-L-AAS |
| 6 | PCS-L-AAS-L |
| 7 | PCS-AAS-L |
| 8 | L-AAS-PCS |
| 9 | L-AAS-L-PCS |
| 10 | L-AAS-PCS-L |
| 11 | L-AAS-L-PCS-L |
| 12 | AAS-L-PCS |
| 13 | AAS-L-PCS-L |
| 14 | AAS-PCS-L |
| 15 | L-PCS-PCS-AAS |
| 16 | PCS-PCS-AAS-L |
| 17 | L-PCS-PCS-AAS-L |
| 18 | L-PCS-PCS-PCS-AAS |
| 19 | PCS-PCS-PCS-AAS-L |
| 20 | L-PCS-PCS-PCS-AAS-L |
| 21 | L-PCS-PCS-PCS-PCS-AAS |
| 22 | PCS-PCS-PCS-PCS-AAS-L |
| 23 | L-PCS-PCS-PCS-PCS-AAS-L |
| 24 | L-AAS-PCS-PCS |
| 25 | AAS-PCS-PCS-L |
| 26 | L-AAS-PCS-PCS-L |
| 27 | L-AAS-PCS-PCS-PCS |
| 28 | AAS-PCS-PCS-PCS-L |
| 29 | L-AAS-PCS-PCS-PCS-L |
| 30 | L-AAS-PCS-PCS-PCS-PCS |
| 31 | AAS-PCS-PCS-PCS-PCS-L |
| 32 | L-AAS-PCS-PCS-PCS-PCS-L |

Abbreviations for Table 5:
L = Linker
PCS = Protease Cleavage Site
AAS = Amino Acid Segment such as a spytag, spycatcher, snooptag, snoopcatcher, or SEQ ID NO: 1 (or a variant thereof) sequence

In some cases, an AAV described herein can be designed to include, within a VP3 polypeptide in an N-terminal to C-terminal direction, a VP3 amino acid sequence immediately followed by a first linker immediately followed by a protease cleavage site immediately followed by a heterologous amino acid segment (e.g., a heterologous amino acid segment having the ability to bind to a binding partner) immediately followed by a second linker immediately followed a continuation of the VP3 amino acid sequence. In another example, an AAV described herein can be designed to include, within a VP3 polypeptide in an N-terminal to C-terminal direction, a VP3 amino acid sequence immediately followed by a first linker followed within 10 amino acid residues by a protease cleavage site immediately followed by a heterologous amino acid segment (e.g., a heterologous amino acid segment having the ability to bind to a binding partner) followed within 10 amino acid residues by a second linker immediately followed a continuation of the VP3 amino acid sequence. In another example, an AAV described herein can be designed to include, within a VP3 polypeptide in an N-terminal to C-terminal direction, a VP3 amino acid sequence immediately followed by a first linker immediately followed by a continuation of the VP3 amino acid sequence that is less than 20 amino acid residues in length immediately followed by a protease cleavage site immediately followed by a heterologous amino acid segment (e.g., a heterologous amino acid segment having the ability to bind to a binding partner) immediately followed a continuation of the VP3 amino acid sequence that is less than 20 amino acid residues in length immediately followed by a second linker immediately followed a continuation of the VP3 amino acid sequence.

An AAV having (a) a VP3 polypeptide that includes a heterologous amino acid segment as described herein and (b) VP1 and VP2 polypeptides that lack that heterologous amino acid segment can have the ability to form a covalent bond with another moiety via that heterologous amino acid segment. For example, an AAV provided herein having a VP3 polypeptide that includes a heterologous amino acid segment as described herein can covalently bind to a polypeptide (e.g., a conjugating polypeptide) or another virus (e.g., another AAV) via that heterologous amino acid segment. To form that covalent bond, the other moiety can be designed to include a binding partner having the ability to form that covalent bond with the heterologous amino acid segment.

As described herein, an AAV having (a) a VP3 polypeptide that includes a heterologous amino acid segment as described herein and (b) VP1 and VP2 polypeptides that lack that heterologous amino acid segment can be covalently bound to another moiety of interest via that heterologous amino acid segment. Any appropriate moiety of interest (e.g., a polypeptide or a virus) can be designed to include a binding partner such that that moiety of interest can be covalently bound to an AAV provided herein via a covalent bond formed between the heterologous amino acid segment of the AAV and the binding partner of the moiety of interest. Examples of moieties of interest that can be designed to include a binding partner described herein and used as described herein include, without limitation, polypeptides such as a conjugating polypeptide, a Rabies G-protein, or a genome editing polypeptide (e.g. a Cas9 polypeptide) and viruses such as AAVs or adenoviruses.

In some cases, a first AAV can be designed to include (a) a VP3 polypeptide that includes a heterologous amino acid segment as described herein and (b) VP1 and VP2 polypeptides that lack that heterologous amino acid segment, and a second AAV can be designed to include a capsid polypeptide (e.g., a VP3 polypeptide) having a binding partner sequence having the ability to covalently bind to the heterologous amino acid segment of the VP3 polypeptide of the first AAV. In such a case, the first AAV can be covalently bound to the second AAV via the heterologous amino acid segment of the VP3 polypeptide of the first AAV and the binding partner sequence of a capsid polypeptide (e.g., a VP1, VP2, and/or VP3 polypeptide) of the second AAV.

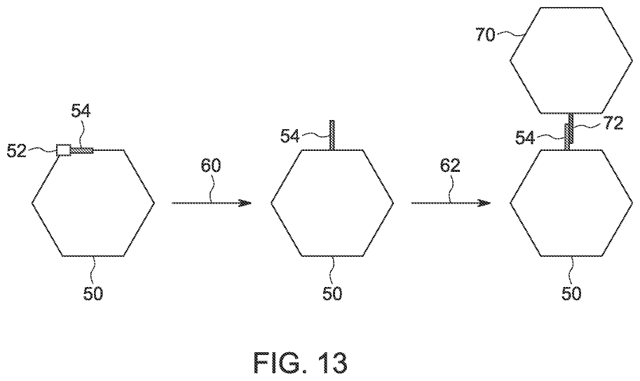

For example, with reference to FIG. 13, a first AAV (50) can be designed to include a VP3 polypeptide containing an amino acid segment (54) (e.g., a spytag sequence) having the ability to bind to (e.g., to covalently bind to) a binding partner (72) (e.g., a spycatcher sequence). A second AAV (70) can be designed to include a capsid polypeptide containing a binding partner (72). First AAV (50) and second AAV (70) can be combined to covalently link first AAV (50) to second AAV (70) via the covalent bond between amino acid segment (54) and binding moiety (72). In some examples, the covalently attached AAVs can enter a single cell as one unit (first AAV (50) and second AAV (70)), thereby delivering the nucleic acid content of the two AAVs to that cell.

In some cases, with further reference to FIG. 13, first AAV (50) can be designed to include a VP3 polypeptide containing a protease cleavage site (52) N-terminal (or C-terminal) to amino acid segment (54). Protease cleavage site (52) of first AAV (50) can be cleaved when exposed to a protease under conditions sufficient to allow proteolytic cleavage (60). Proteolytic cleavage (60) of protease cleavage site (52) can produce amino acid segment (54) where the N-terminal end (or C-terminal end) of amino acid segment (54) is released from the VP3 polypeptide. Released amino acid segment (54) can more readily form a covalently bond with binding partner (72) on second AAV (70).

In some cases, with further reference to FIG. 13, second AAV (70) can be designed to include a capsid polypeptide (e.g., VP1, VP2, and/or VP3 polypeptide(s)) containing a protease cleavage site N-terminal (or C-terminal) to binding partner (72). In these cases, proteolytic cleavage of the protease cleavage site can produce binding partner (72) where the N-terminal end (or C-terminal end) of binding partner (72) is released from the capsid polypeptide. A released binding partner (72) can more readily form a covalent bond with amino acid segment (54).

In some cases, AAVs (e.g., the “second AAV” of the preceding two paragraphs) having a capsid polypeptide designed to include a binding partner having the ability to covalently bind to the heterologous amino acid segment of another AAV (e.g., the “first AAV”) can be designed such that all three of VP1, VP2, and VP3 contains the introduced binding partner. In other cases, those AAVs (e.g., the “second AAV” of the preceding two paragraphs) can be designed to have (a) a VP3 polypeptide that includes the binding partner sequence and (b) VP1 and VP2 polypeptides that lack that binding partner sequence.

In another example, an AAV can be designed to include (a) a VP3 polypeptide that includes a heterologous amino acid segment as described herein and (b) VP1 and VP2 polypeptides that lack that heterologous amino acid segment, and a polypeptide (e.g., a conjugating polypeptide) can be designed to include a binding partner sequence having the ability to covalently bind to the heterologous amino acid segment of VP3 polypeptide of the AAV. In such a case, the AAV can be covalently bound to the polypeptide (e.g., a conjugating polypeptide) via the heterologous amino acid segment of the VP3 polypeptide of the AAV and the binding partner of the polypeptide (e.g., a conjugating polypeptide). Such a polypeptide (e.g., a conjugating polypeptide) containing a binding partner sequence having the ability to covalently bind to the heterologous amino acid segment that is included within a VP3 polypeptide of an AAV as described herein can be used to covalently attach molecules of interest to that AAV via that polypeptide (e.g., conjugating polypeptide). For example, a conjugating polypeptide can be designed to include an enzyme (i.e., a molecule of interest in this example) and a binding partner sequence having the ability to covalently bind to a heterologous amino acid segment of a VP3 polypeptide of an AAV. In this example, such a conjugating polypeptide can be used to covalently attach that enzyme to the surface of the AAV that includes the VP3 polypeptide having the heterologous amino acid segment capable of binding to the binding partner of the conjugating polypeptide.

In some cases, a conjugating polypeptide can be designed to include two or more binding partners having the ability to covalently bind to a heterologous amino acid segment. In such cases, the two or more binding partners can be identical or different. For example, a conjugating polypeptide can be designed to include a first binding partner having the ability to covalently bind to a first heterologous amino acid segment and a second binding partner having the ability to covalently bind to a second heterologous amino acid segment that is the same or different from the first heterologous amino acid segment. In some cases, a conjugating polypeptide can be designed to include a first binding partner containing a spycather sequence and a second binding partner containing snoopcatcher sequence. In another example, a conjugating polypeptide can be designed to include a first spycather sequence and a second spycatcher sequence. In another example, a conjugating polypeptide can be designed to include a first snoopcather sequence and a second snoopcatcher sequence.

A conjugating polypeptide provided herein and designed to include two or more binding partners each having the ability to covalently bind to a heterologous amino acid segment described herein can be used to covalently link two AAVs together. With reference to FIG. 2, a first AAV (10) can be designed to include a VP3 polypeptide (12) containing a heterologous amino acid segment (14) having the ability to bind to (e.g., to covalently bind to) a binding partner as described herein. First AAV (10) can contain VP1 and VP2 polypeptides that lack heterologous amino acid segment (14). A second AAV (20) can be designed to include a VP3 polypeptide (22) containing a heterologous amino acid segment (24) having the ability to bind to (e.g., to covalently bind to) a binding partner as described herein. In addition, a conjugating polypeptide (30) can be designed to include a first binding partner (32) and a second binding partner (34). In some cases, first binding partner (32) and second binding partner (34) can be linked with a linker sequence (36). Any appropriate linker sequence can be used to link two or more binding partner sequences of a conjugating polypeptide together. Examples of linker sequences that can be used to link two or more binding partner sequences of a conjugating polypeptide together include, without limitation, those set forth in Table 6.

TABLE 6
Example of linker sequences for
conjugating polypeptides.

| Sequence | SEQ ID NO: |
|---|---|
| SGGGGSGGGGSGGGGS | 66 |
| LAEAAAKEAAAKAAA | 67 |
| GSGGGSGGGSPANLKALEAQKQKEQR | 68 |
| QAAEELANAKKLKEQLEKGSGGGSGG | |
| GS | |

With further reference to FIG. 2, first AAV (10), second AAV (20), and conjugating polypeptide (30) can be combined to covalently link first AAV (10) to second AAV (20) via conjugating polypeptide (30). In this example, first AAV (10a) is covalently linked to second AAV (20a) via a first covalent bond between amino acid segment (14a) and first binding partner (32a) and a second covalent bond between amino acid segment (24a) and second binding partner (34a). In some examples, the covalently attached AAVs can enter a single cell as one unit (first AAV (10b) and second AAV (20b)), thereby delivering the nucleic acid content of the two AAVs to that cell. In some cases, first AAV (10b) and second AAV (20b) can contain different genetic material. For example, first AAV (10b) can include nucleic acid (16), and second AAV (20b) can include nucleic acid (26), which is different from that of nucleic acid (16). In some cases, the delivered nucleic from two or more covalently linked AAVs can contain overlapping sequences such that they can recombine to form a lager nucleic acid within the cell. For example, nucleic acid (16) and nucleic acid (26) can contain overlapping sequences such that they can recombine to form a lager nucleic acid shown as nucleic acid (16a) and nucleic acid (26a).

In some cases, a conjugating polypeptide provided herein can be designed to include a first binding partner having the ability to covalently bind to a heterologous amino acid segment containing the amino acid sequence set forth in SEQ ID NO:1 (or a variant thereof) and a second binding partner having the ability to covalently bind to a spytag sequence or a snooptag sequence. In these cases, the conjugating polypeptide can be used to covalently link a first AAV that includes a VP3 polypeptide having a heterologous amino acid segment containing the amino acid sequence set forth in SEQ ID NO:1 (or a variant thereof) to a second AAV that includes a VP3 polypeptide having that spytag sequence or snooptag sequence.

This document also provides compositions containing an AAV described herein. For example, compositions can be produced to contain one or more AAVs having (a) VP3 polypeptides that include a heterologous amino acid segment (e.g., a heterologous amino acid segment having the ability to bind to a binding partner) and (b) VP1 and VP2 polypeptides that lack that heterologous amino acid segment as described herein. In some cases, such a composition can include the AAVs at a titer that is greater than 10¹¹ (e.g., greater than 1×10¹¹, greater than 2×10¹¹, greater than 3×10¹¹, greater than 4×10¹¹, greater than 5×10¹¹, greater than 6×10¹¹, greater than 7×10¹¹, greater than 8×10¹¹, or greater than 9×10¹¹, greater than 1×10¹², greater than 2×10¹², greater than 3×10¹², greater than 4×10¹², greater than 5×10¹², greater than 6×10¹², greater than 7×10¹², greater than 8×10¹², or greater than 9×10¹², greater than 1×10¹³, greater than 2×10¹³, greater than 3×10¹³, greater than 4×10¹³, greater than 5×10¹³, greater than 6×10¹³, greater than 7×10¹³, greater than 8×10¹³, or greater than 9×10¹³, greater than 1×10¹⁴, greater than 2×10¹⁴, greater than 3×10¹⁴, greater than 4×10¹⁴, greater than 5×10¹⁴, greater than 6×10¹⁴, greater than 7×10¹⁴, greater than 8×10¹⁴, or greater than 9×10¹⁴). In some cases, a composition provided herein can have AAVs with a titer that is greater than (e.g., at least 5-fold, 10-fold, 25-fold, 50-fold, 75-fold, or 100-fold greater than) the titer of an AAV preparation of a comparable AAV that contains VP1, VP2, and VP3 polypeptides having the heterologous amino acid segment. For example, a composition provided herein having an AAV designed to have (a) VP3 polypeptides that include a heterologous amino acid segment (e.g., a heterologous amino acid segment having the ability to bind to a binding partner) and (b) VP1 and VP2 polypeptides that lack that heterologous amino acid segment can have an AAV titer that is greater than (e.g., at least 5-fold, 10-fold, 25-fold, 50-fold, 75-fold, or 100-fold greater than) an AAV preparation of a comparable AAV that contains VP1, VP2, and VP3 polypeptide having the heterologous amino acid segment.

This document also provides nucleic acid molecules and vector systems for producing an AAV having (a) a VP3 polypeptide that includes a heterologous amino acid segment (e.g., a heterologous amino acid segment having the ability to bind to a binding partner) and (b) VP1 and VP2 polypeptides that lack that heterologous amino acid segment. For example, a first isolated nucleic acid molecule provided herein (e.g., an AAV trans vector for VP3) can be designed to encode (a) a VP1 polypeptide, (b) a VP2 polypeptide, and (c) a VP3 polypeptide containing a heterologous amino acid segment (e.g., a heterologous amino acid segment having the ability to bind to a binding partner); except that the start codon of the VP1 polypeptide is deleted and except that a stop codon is inserted into the coding frame of the VP2 sequence between the VP2 start codon and the VP3 start codon. In such cases, the start codon of the VP1 sequence can be changed from ATG to any other codon except a stop codon. For example, the start codon of the VP1 sequence can be changed from ATG to ATC, CTT, or TCT. In addition, the stop codon inserted into the coding frame of the VP2 sequence between the VP2 start codon and the VP3 start codon can be any appropriate stop codon (e.g., TAA, TGA, or TAG), and the stop codon can be inserted at any appropriate location between the VP2 start codon and the VP3 start codon. For example, a stop codon can be inserted between 138 and 202 codons before the VP3 start codon. This vector can be used to produce a VP3 polypeptide having a heterologous amino acid segment (e.g., a heterologous amino acid segment having the ability to bind to a binding partner).

A second isolated nucleic acid molecule provided herein (e.g., an AAV trans vector for VP1 and VP2) can be designed to encode (a) a VP1 polypeptide (e.g., a wild-type VP1 polypeptide), (b) a VP2 polypeptide (e.g., a wild-type VP2 polypeptide), and (c) a VP3 polypeptide (e.g., a wild-type VP3 polypeptide); except that the start codon of the VP3 polypeptide is deleted. In this case, the vector can lack the sequence encoding a heterologous amino acid segment having the ability to bind to a binding partner. The start codon of the VP3 sequence can be changed from ATG to any other codon except a stop codon. For example, the start codon of the VP3 sequence can be changed from ATG to ATC, CTT, or TCT. This vector can be used to produce a VP1 polypeptide and a VP2 polypeptide with each not containing a heterologous amino acid segment having the ability to bind to a binding partner.

This document also provides conjugating polypeptides (and nucleic acids encoding conjugating polypeptides) as described herein. For example, conjugating polypeptides provided herein can be designed to contain one or more binding partner sequences (e.g., one binding partner sequence or two binding partner sequences) having the ability to bind covalently to heterologous amino acid segment(s) of an AAV provided herein. In some cases, conjugating polypeptides are provided herein that include a binding partner sequence having the ability to form a covalent bond with an amino acid segment containing the amino acid sequence set forth in SEQ ID NO:1 (or a variant thereof), a spytag sequence, or a snooptag sequence.

In some cases, a conjugating polypeptide provided herein can include two or more binding partner sequences where at least one binding partner includes the ability to bind covalently to the amino acid segment set forth in SEQ ID NO:1 (or a variant thereof). For example, a conjugating polypeptides provided herein can include a first binding partner having the ability to bind covalently to the amino acid segment set forth in SEQ ID NO:1 (or a variant thereof) and a second binding partner having the ability to bind covalently to the amino acid segment set forth in SEQ ID NO:1 (or a variant thereof). In another example, a conjugating polypeptide can include a first binding partner having the ability to bind covalently to the amino acid segment set forth in SEQ ID NO:1 (or a variant thereof) and a second binding partner having the ability to covalently bind to a spytag sequence or a snooptag sequence. In another example, a conjugating polypeptide can include a first binding partner having the ability to bind covalently to a spytag sequence and a second binding partner having the ability to covalently bind to a snooptag sequence. In another example, a conjugating polypeptide can include a first binding partner having the ability to bind covalently to a spytag sequence and a second binding partner having the ability to covalently bind to a spytag sequence. In another example, a conjugating polypeptide can include a first binding partner having the ability to bind covalently to a snooptag sequence and a second binding partner having the ability to covalently bind to a snooptag sequence.

In some cases, a conjugating polypeptides provided herein can include a linker (e.g., any of the exemplary linkers described herein) located between the two binding partners. In some cases, the two or more binding partner sequences can directly abut each other in the conjugating polypeptide.

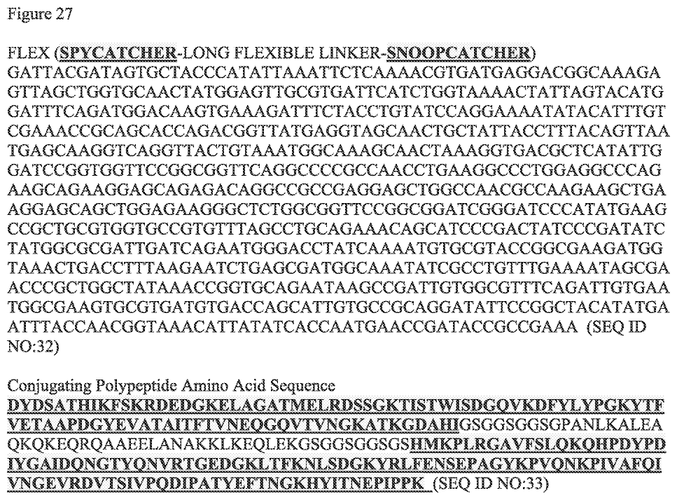

Also provided in this document are nucleic acid molecules encoding any of the exemplary conjugating polypeptides described herein. For example, isolated nucleic acid molecules provided herein can encode for a conjugating polypeptide that includes a binding partner (e.g., any of the exemplary binding partners described herein) that binds covalently to a heterologous amino acid segment present in a VP3 polypeptide of an AAV. In such cases, a nucleic acid molecule provided herein can encode the amino acid sequence set forth in FIG. 27, 28, or 29.

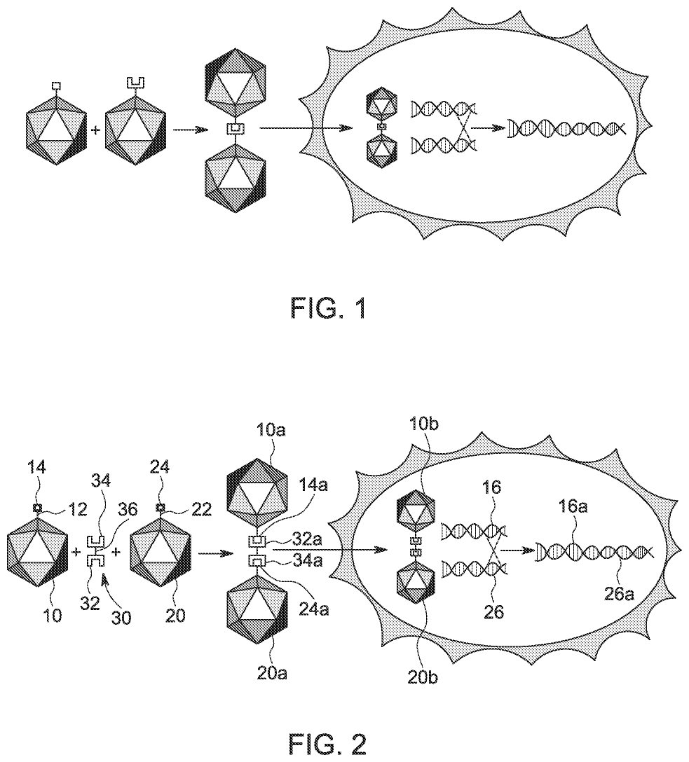

In addition, this document provides methods for making a composition that includes two or more different AAVs covalently linked together. For example, compositions can be produced to contain two or more AAVs linked together through one or more covalent bonds (see, e.g., FIG. 1 or 2). In some cases, a covalent linkage can be provided by an interaction between a first AAV having (a) a VP3 polypeptide that includes a heterologous amino acid segment as described herein and (b) VP1 and VP2 polypeptides lacking that heterologous amino acid sequence, and a second AAV having a capsid polypeptide (e.g., a VP1, VP2, and/or VP3 polypeptide) that includes a binding partner for the heterologous amino acid segment of the VP3 polypeptide of the first AAV (FIG. 1). For example, a second AAV can include a VP3 polypeptide that includes a binding partner having the ability to bind to the heterologous amino acid segment of the VP3 polypeptide of the first AAV and can include VP1 and VP2 polypeptides that lack that binding partner.

In some cases, a covalent linkage can be provided by an interaction between a first AAV having (a) a VP3 polypeptide that includes a heterologous amino acid segment as described herein and (b) VP1 and VP2 polypeptides lacking that heterologous amino acid sequence and a conjugating polypeptide, and an interaction between that conjugating polypeptide and a second AAV having a capsid polypeptide (e.g., a VP1, VP2, and/or VP3 polypeptide) that includes a heterologous amino acid segment (FIG. 2). In some cases, the second AAV can include a VP3 polypeptide that includes a heterologous amino acid segment having the ability to bind a binding partner of the conjugating polypeptide and can include VP1 and VP2 polypeptides that lack that heterologous amino acid segment.

In some cases, this document provides methods for generating a composition including a first AAV covalently linked to a second AAV. Such methods can include providing (i) a first AAV having (a) a VP3 polypeptide that includes a heterologous amino acid segment as described herein (e.g., a spytag sequence) and (b) VP1 and VP2 polypeptides lacking that heterologous amino acid sequence and (ii) a second AAV including a capsid polypeptide containing a binding partner (e.g., spycatcher sequence). Such a method can then include contacting the first AAV with the second AAV to form a “first AAV-second AAV” complex (see, e.g., FIG. 1).

In some cases, this document provides methods for generating a composition including a first AAV covalently linked to a second AAV via a conjugating polypeptide. Such methods can include providing (i) a first AAV having (a) a VP3 polypeptide that includes a heterologous amino acid segment as described herein (e.g., a spytag sequence) and (b) VP1 and VP2 polypeptides lacking that heterologous amino acid sequence, (ii) a second AAV including a capsid polypeptide (e.g., a VP1, VP2, and/or VP3 polypeptide) containing a heterologous amino acid segment (e.g., a snooptag sequence), and (iii) a conjugating polypeptide having a first binding partner (e.g., a spycatcher sequence) and a second binding partner (e.g., a snoopcatcher sequence). Such a method can then include contacting the first AAV with a conjugating polypeptide to form a first AAV-conjugating polypeptide complex. In such cases, the first AAV-conjugating polypeptide complex can be contacted with the second AAV to form a “first AAV-conjugating polypeptide-second AAV” complex.

In some cases, a composition provided herein can be purified after the formation of a first AAV-conjugating polypeptide complex or after the formation of a second AAV-conjugating polypeptide complex. In such cases, the purification can enrich a solution for an AAV-conjugating polypeptide complex. In some cases, a composition provided herein can undergo a purification step after formation of the “first AAV-conjugating polypeptide-second AAV” complex or after formation of the “second AAV-conjugating polypeptide-first AAV” complex. In such cases, the purification results in a composition enriched for the AAV-conjugating polypeptide-AAV complexes.

The invention will be further described in the following examples, which do not limit the scope of the invention described in the claims.

### EXAMPLES

### Example 1—Construction of AAVs Containing VP3 Polypeptides Having a Heterologous Amino Acid Segment

### Construction of AAV Vectors for Expressing VP3 and not VP1 and VP2

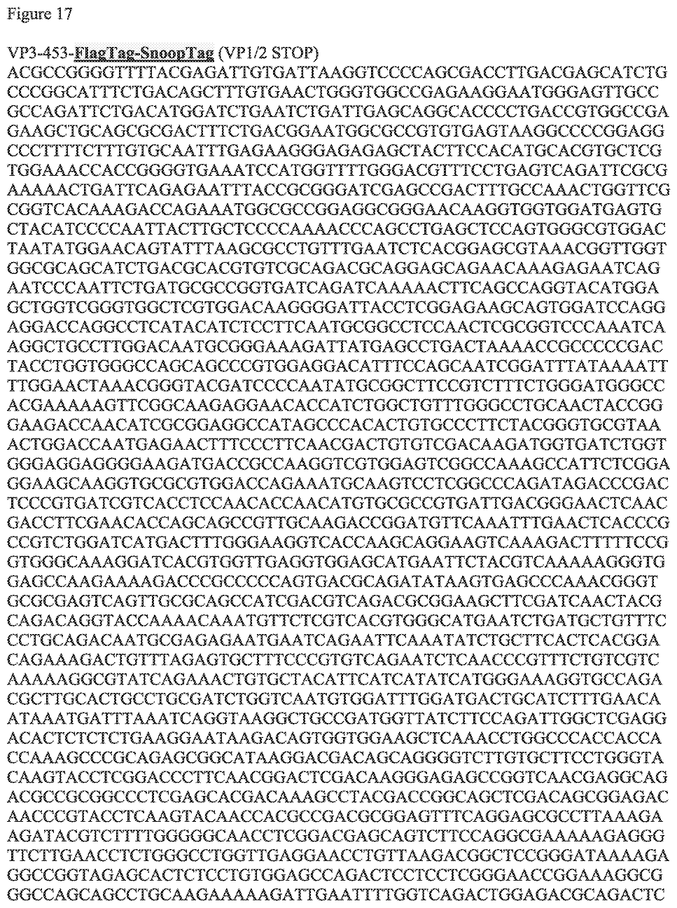

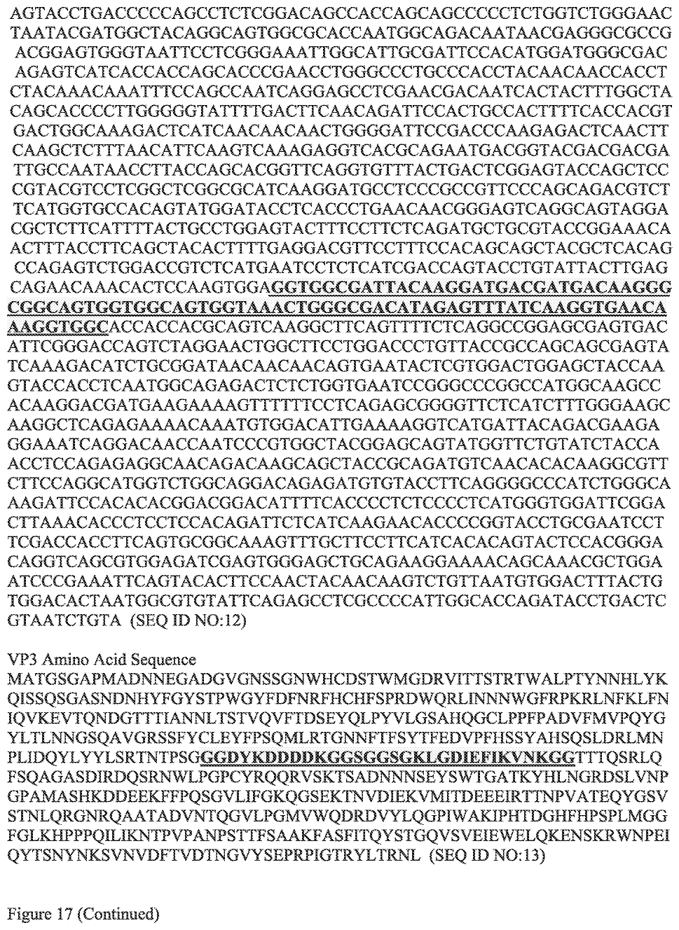

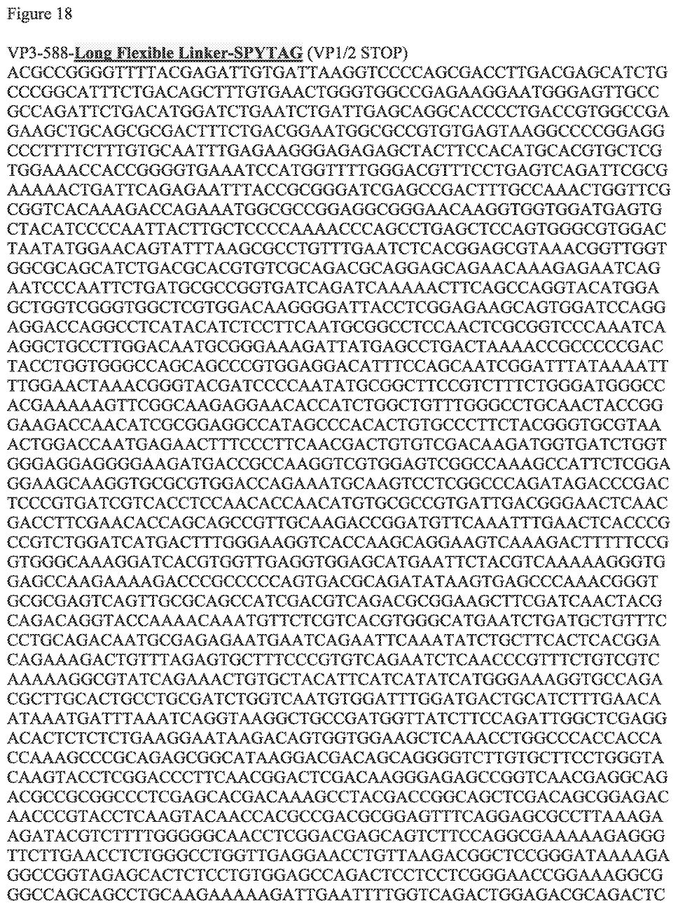

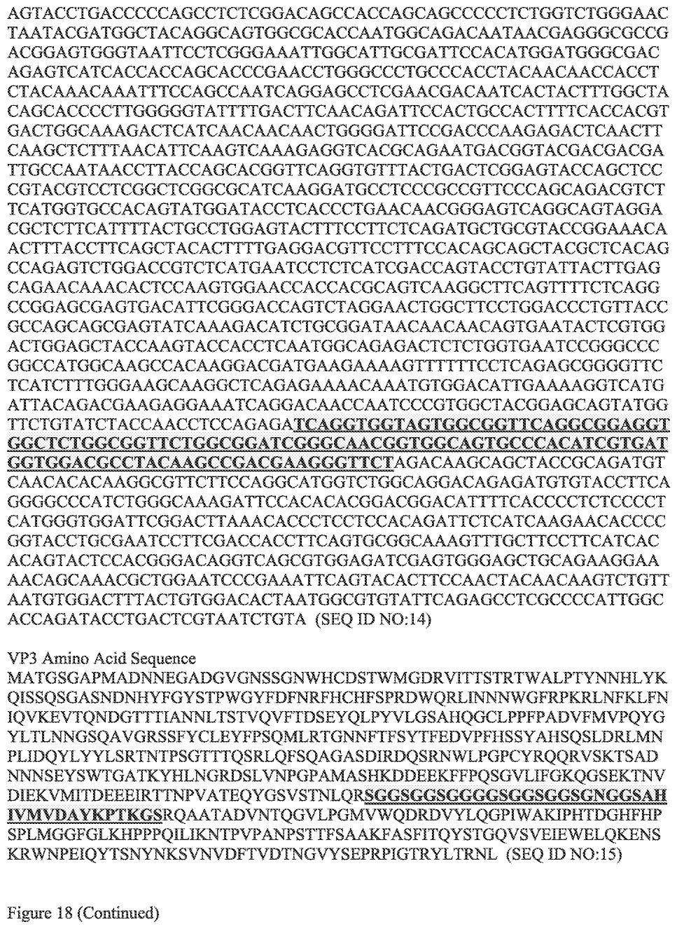

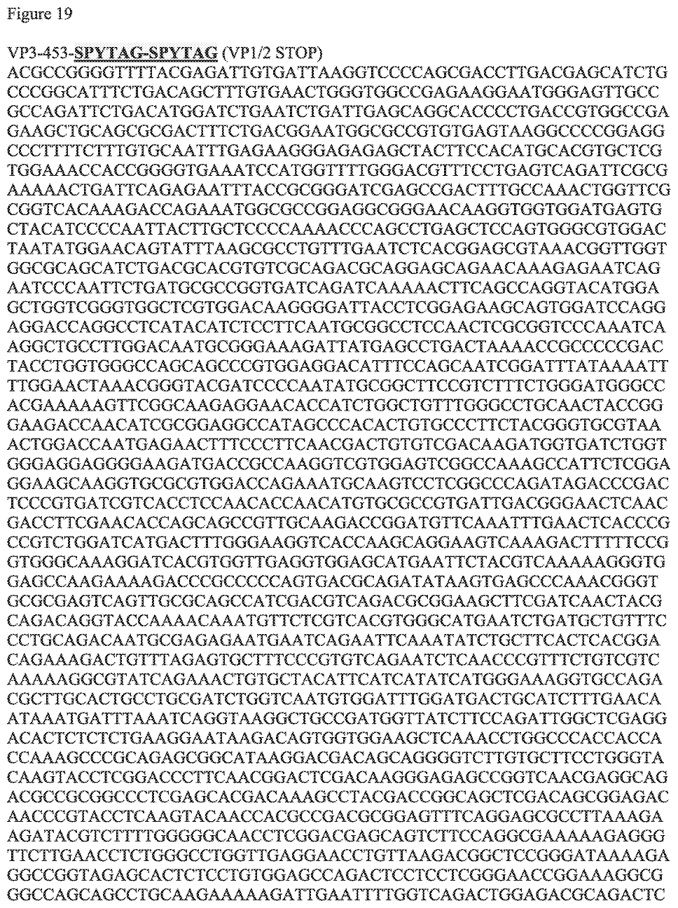

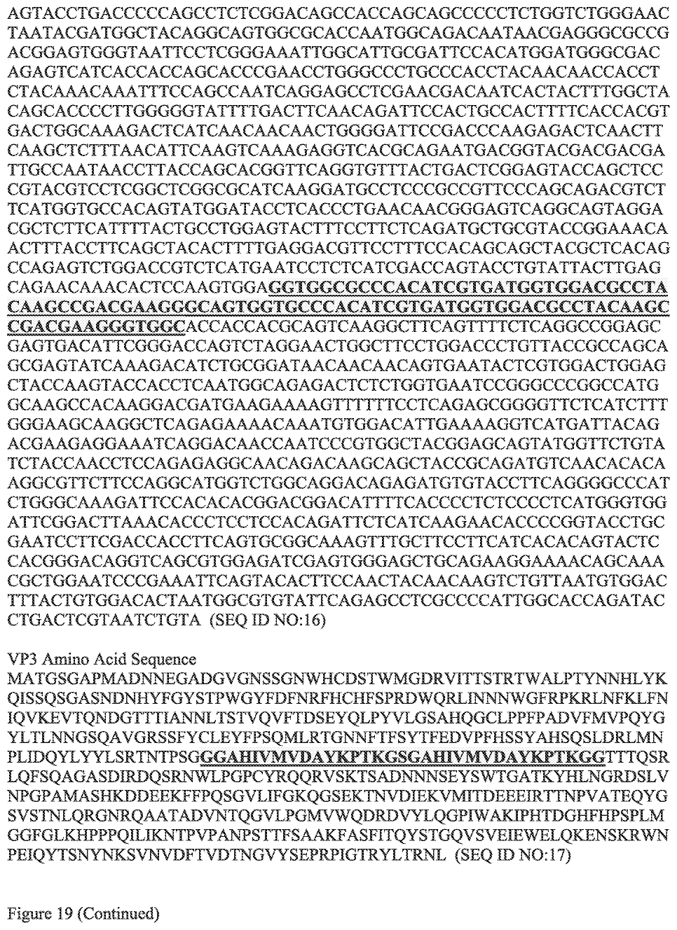

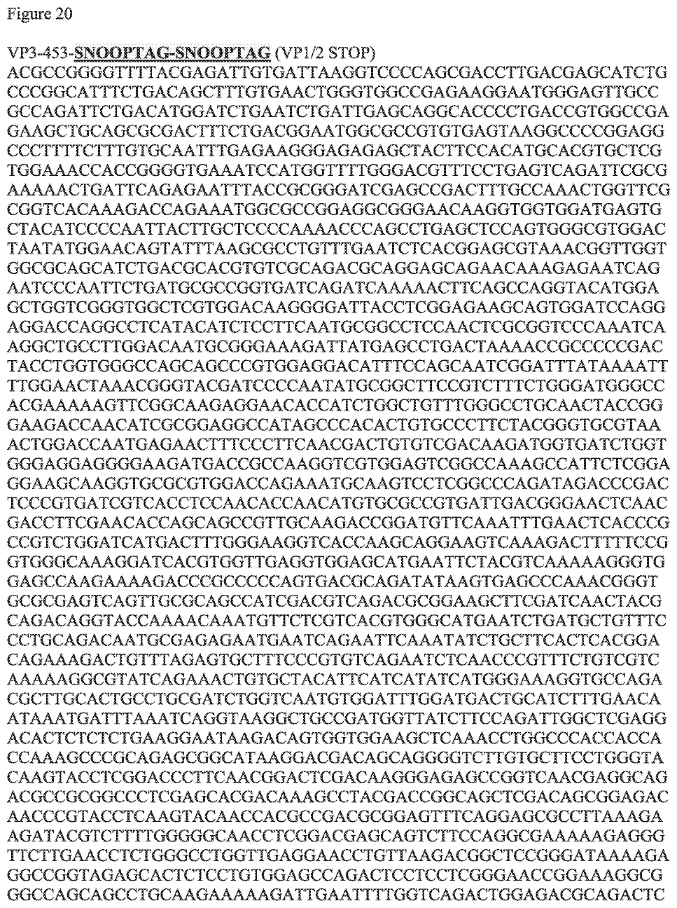

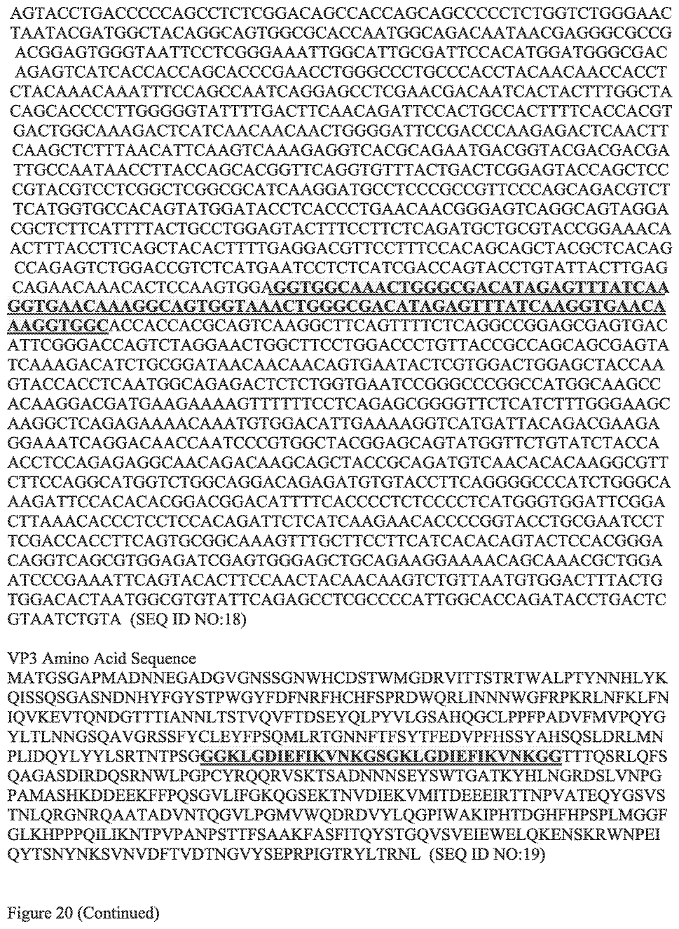

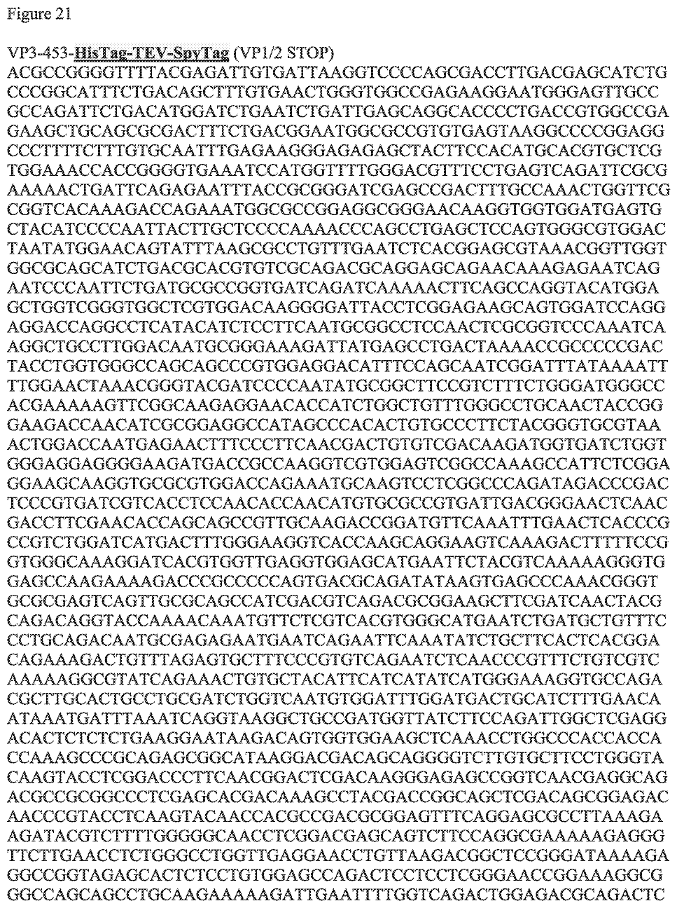

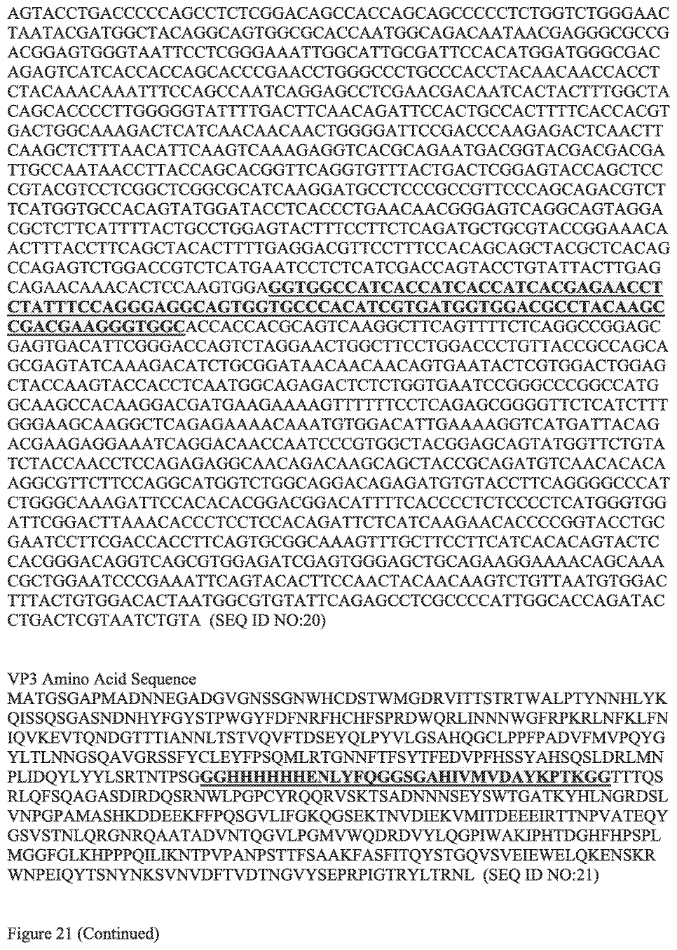

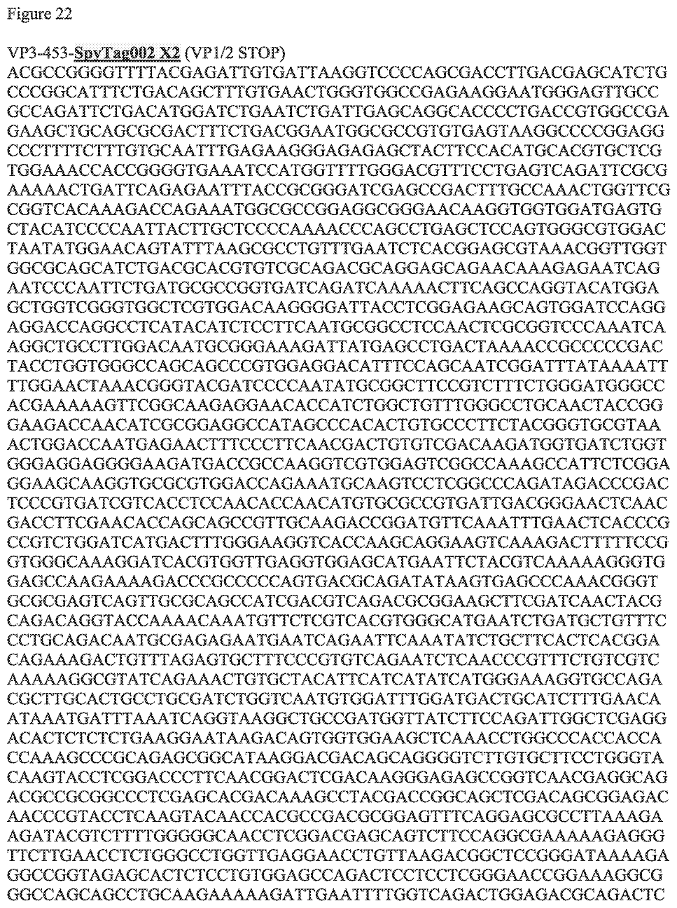

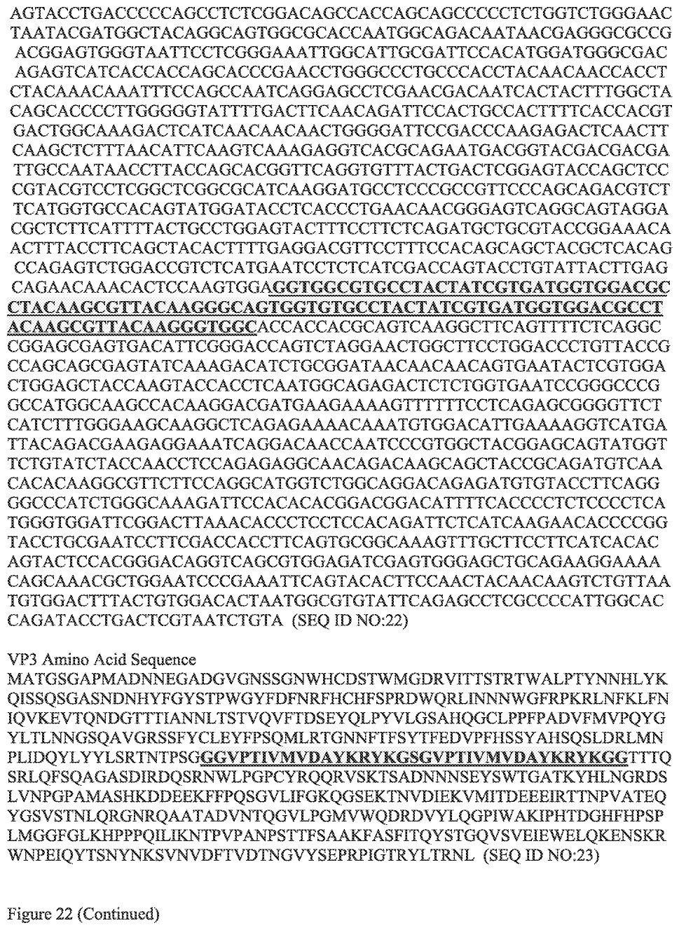

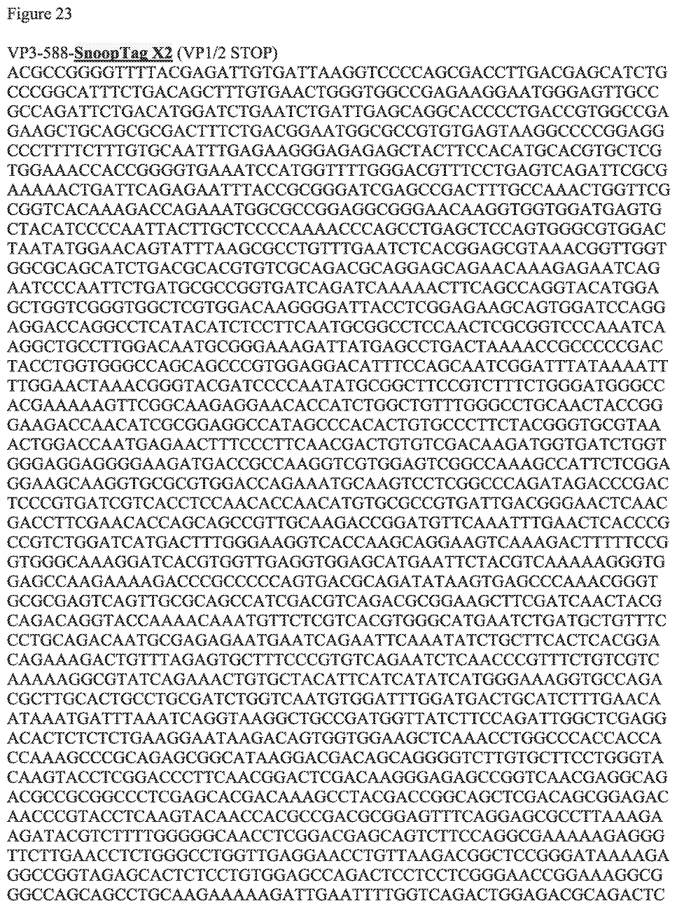

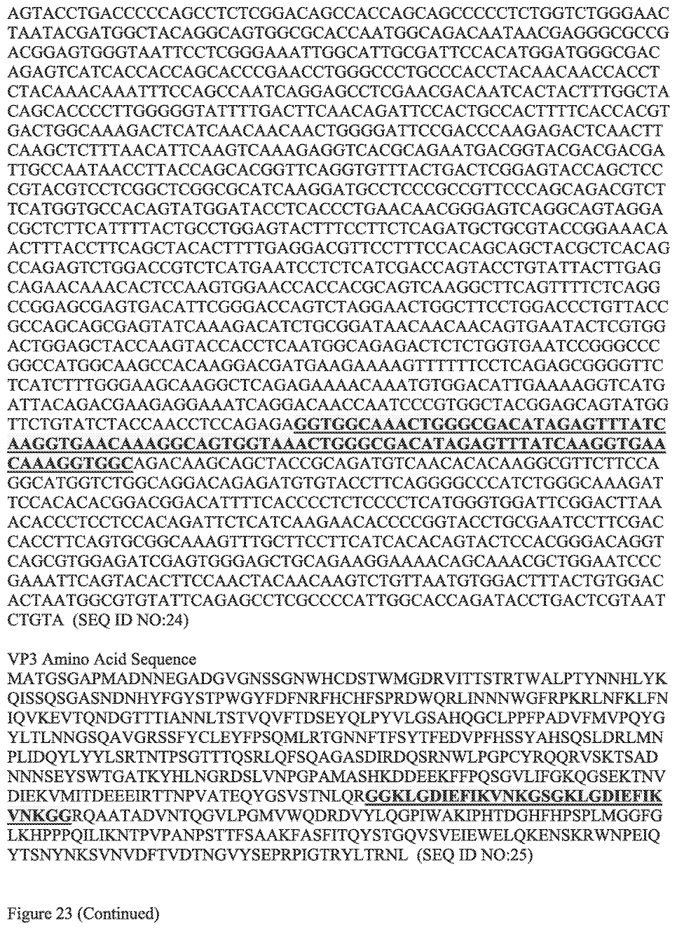

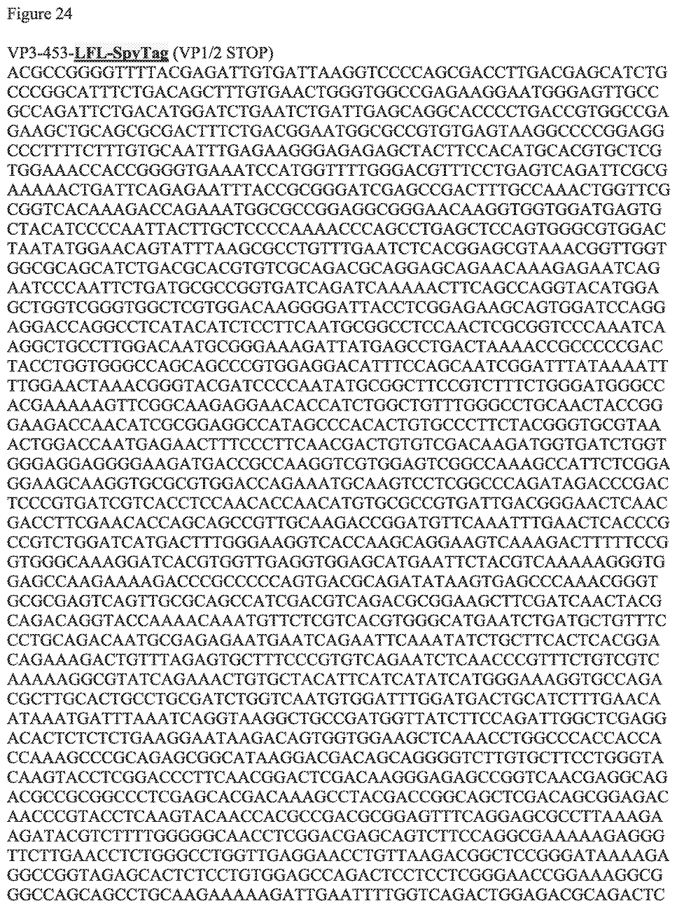

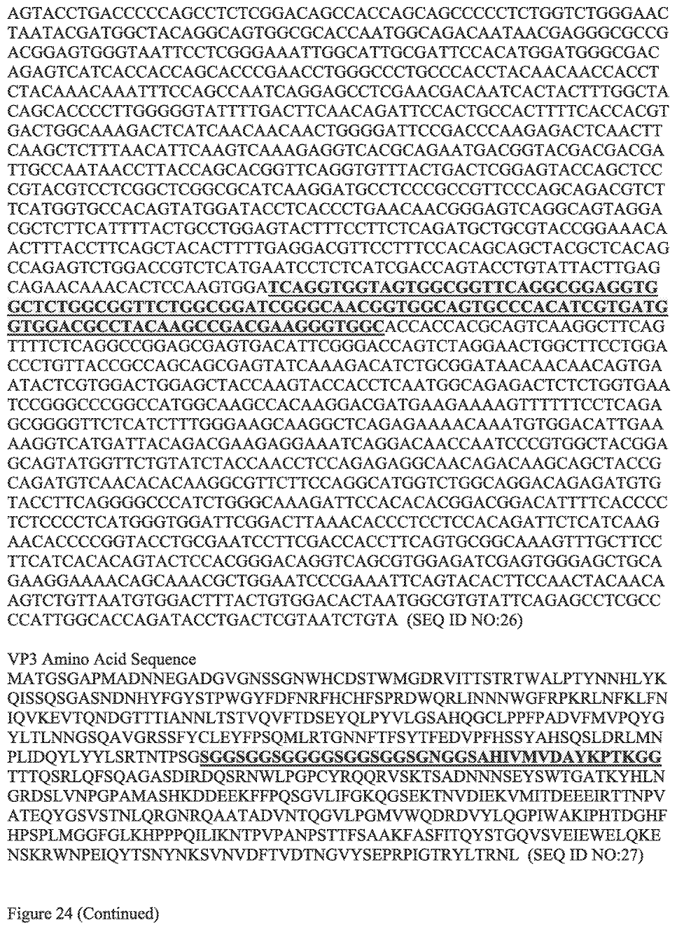

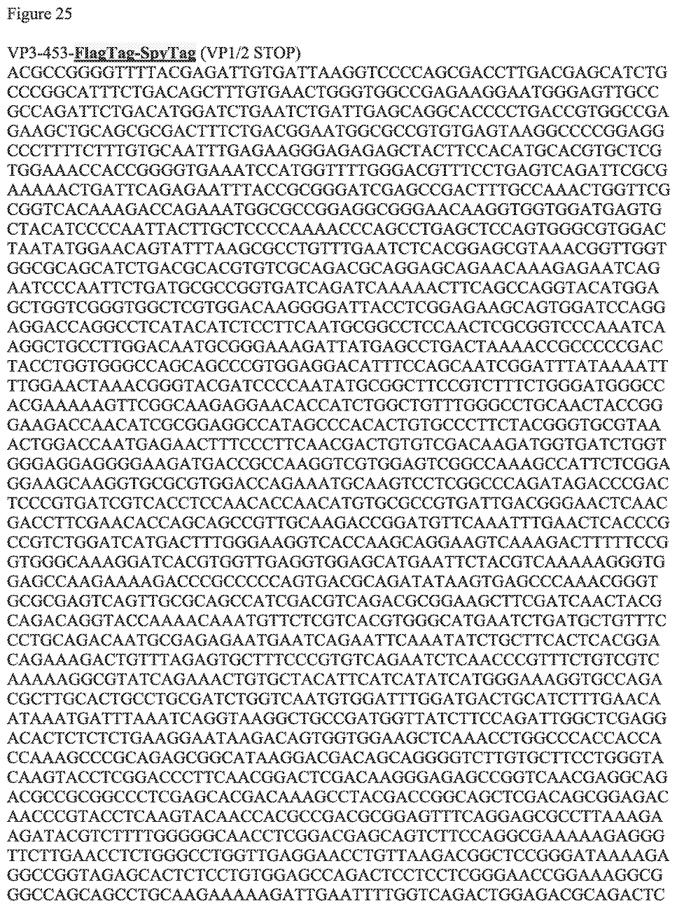

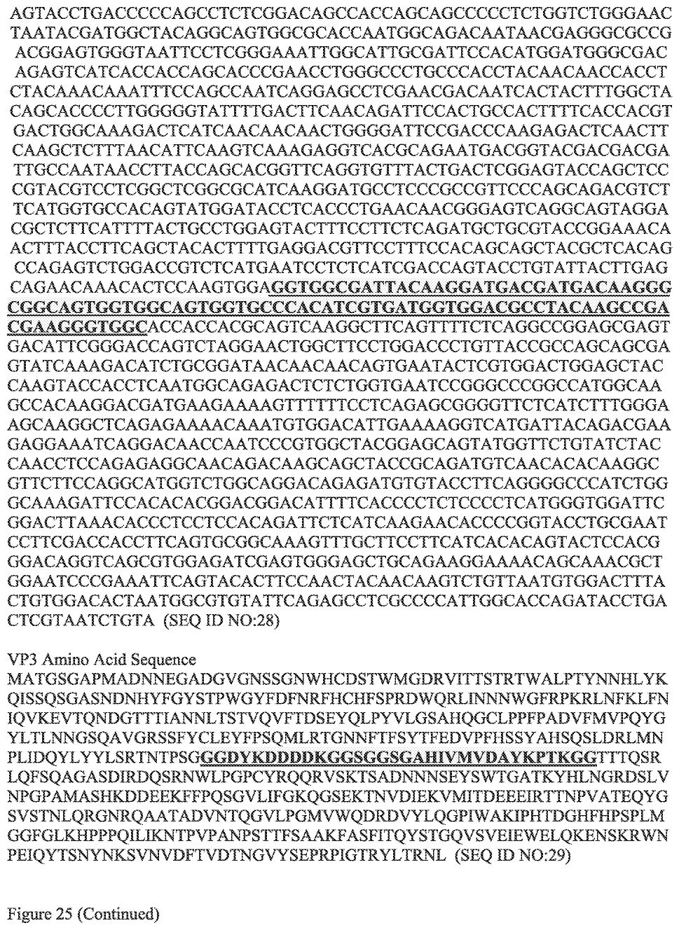

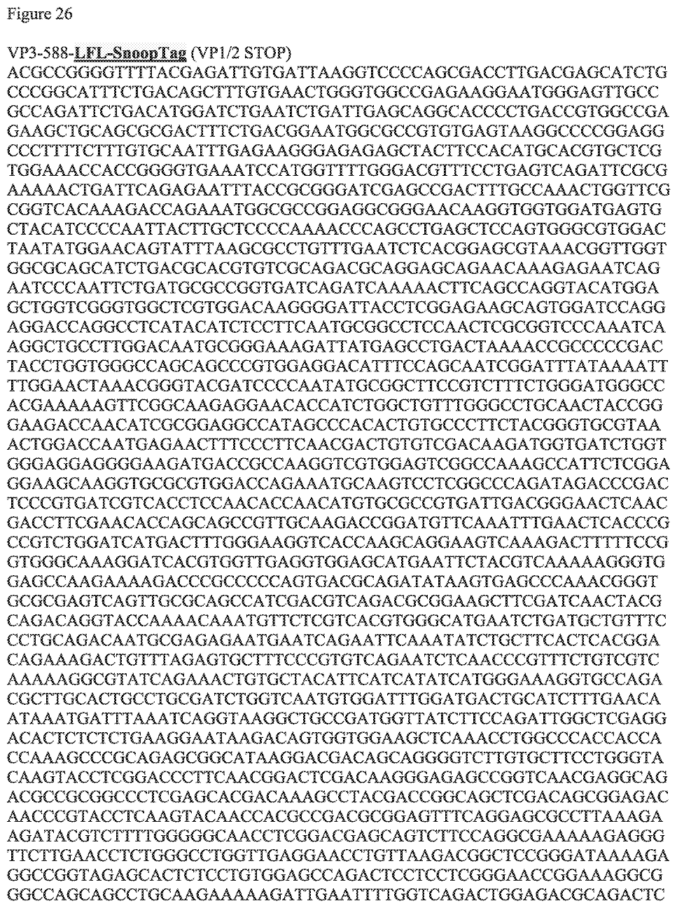

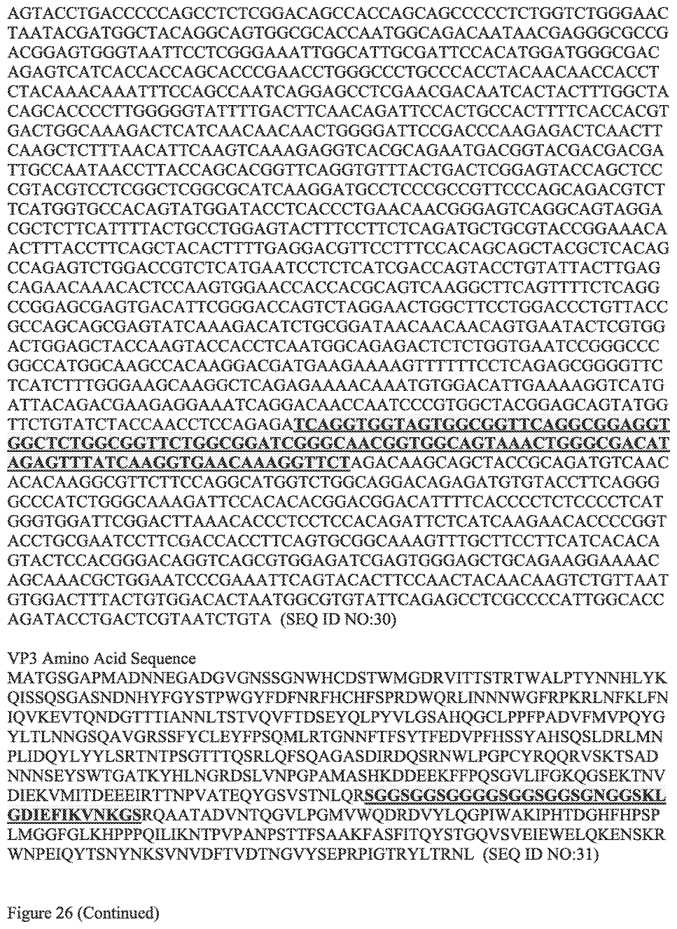

An AAV vector plasmid was mutated so that the start codon of the VP1 polypeptide was deleted and a stop codon was inserted into the coding frame of the VP2 sequence between the VP2 start codon and the VP3 start codon by PCR amplifying the insert regions and annealing with Gibson Assembly (New England Biolabs). Such vectors included the following constructs: VP3-453-FlagTag-SnoopTag (VP1/2 STOP) (FIG. 17), VP3-588-LFL-ST (VP1/2 STOP) (FIG. 18), VP3-453-SpyTag X2 (VP1/2 STOP) (FIG. 19), VP3-453-SnoopTag X2 (VP1/2 STOP) (FIG. 20), VP3-453-HisTag-TEV-SpyTag (VP1/2 STOP) (FIG. 21), VP3-453-SpyTag002 X2 (VP1/2 STOP) (FIG. 22), VP3-588-SnoopTag X2 (VP1/2 STOP) (FIG. 23), VP3-453-LFL-SpyTag (VP1/2 STOP) (FIG. 24), VP3-453-FlagTag-SpyTag (VP1/2 STOP) (FIG. 25), and VP3-588-LFL-SnoopTag (VP1/2 STOP) (FIG. 26).

### Construction of AAV Vectors for Expressing VP1 and VP2 and not VP3

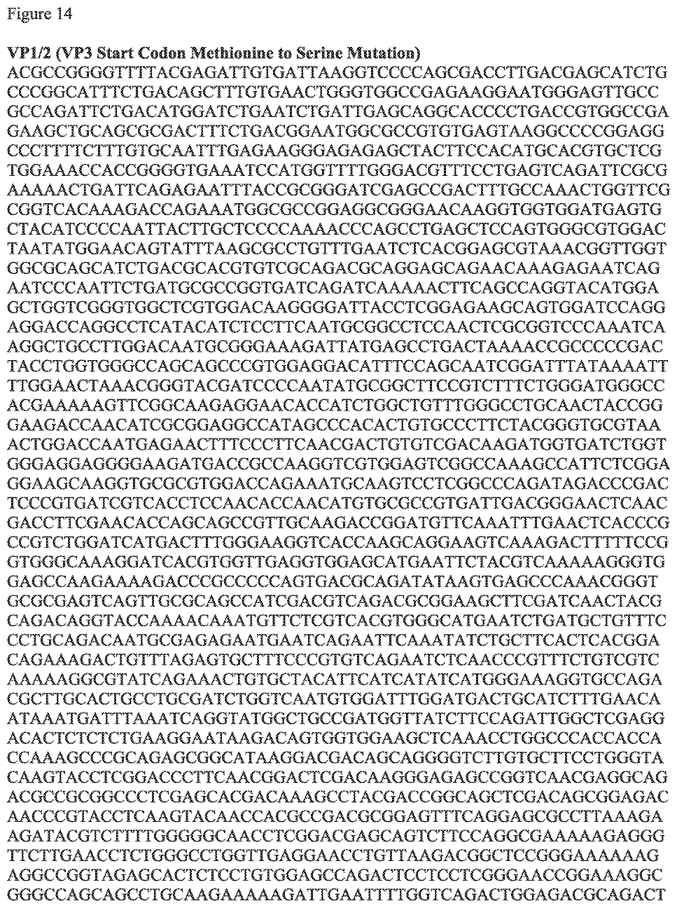

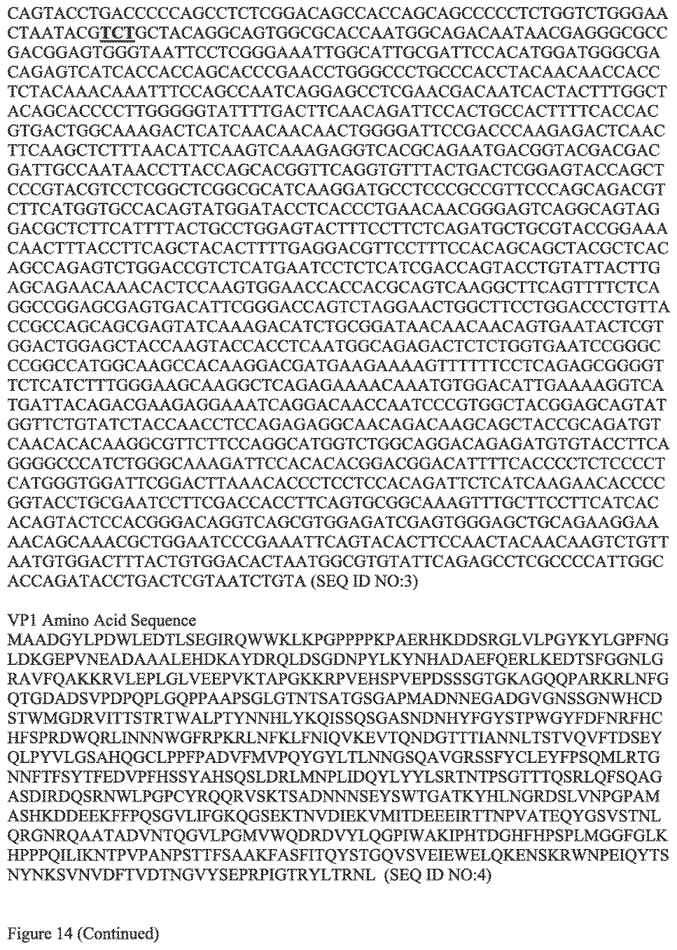

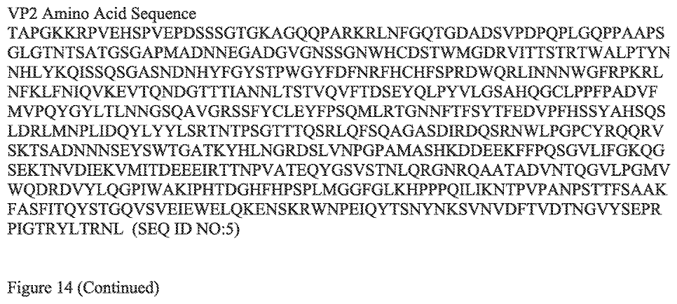

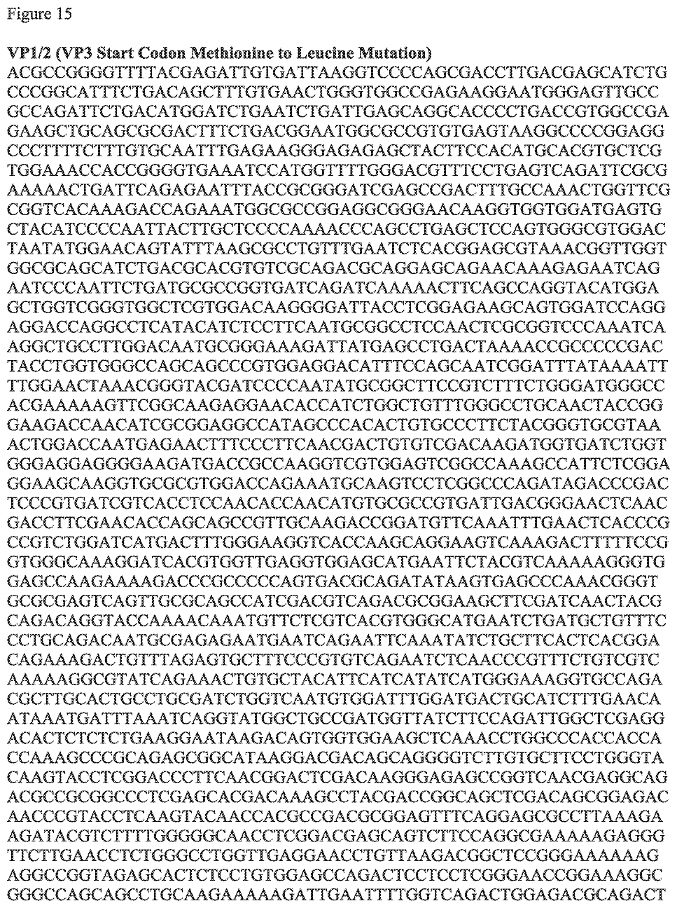

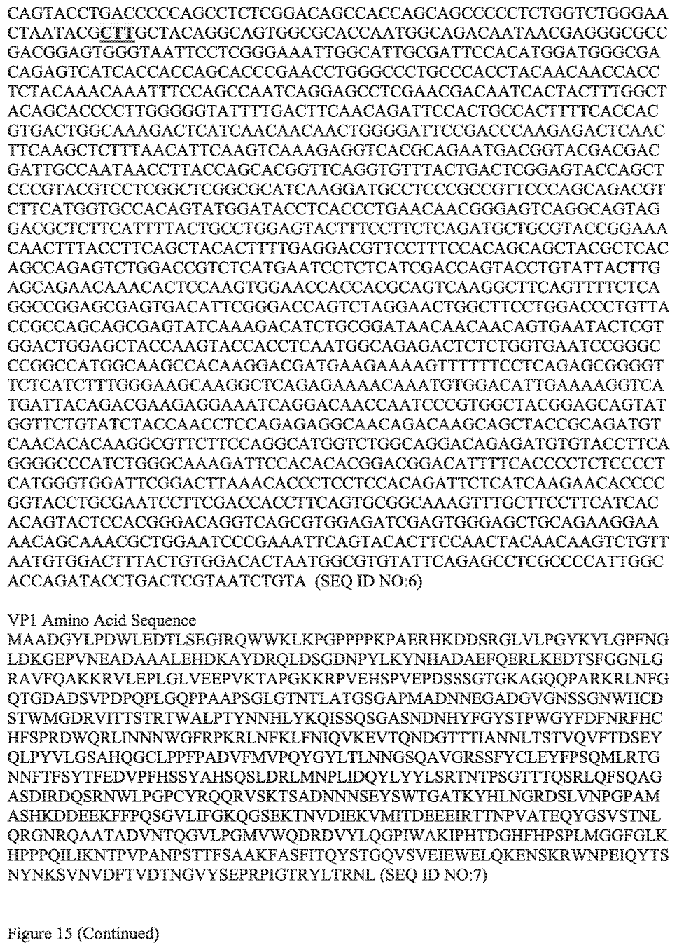

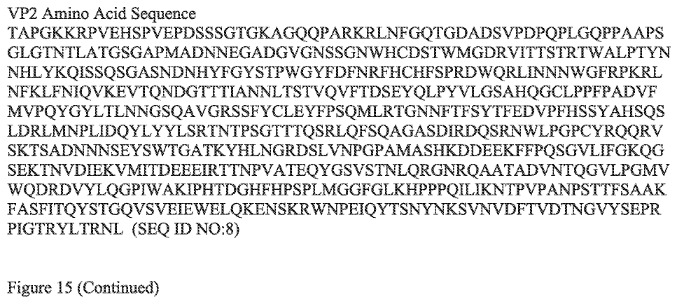

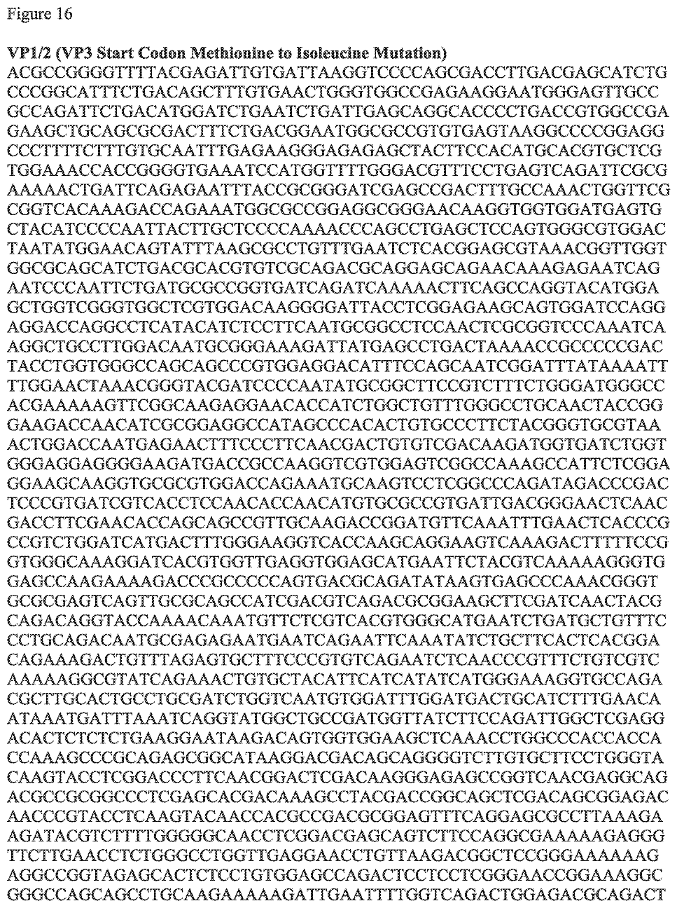

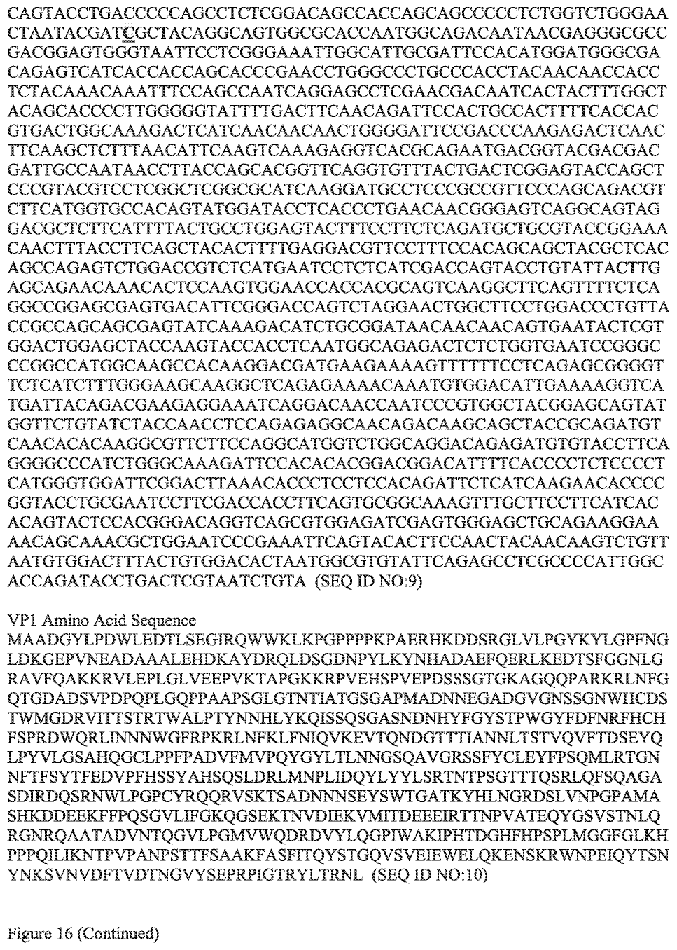

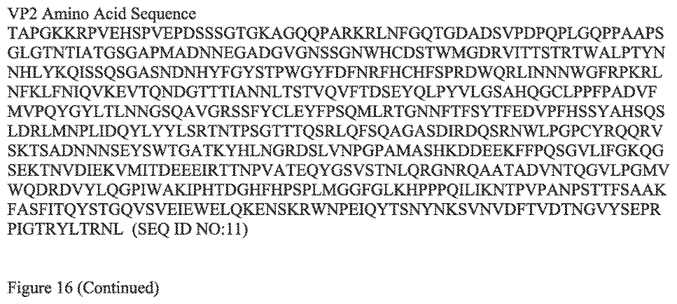

An AAV vector plasmid was mutated so that the start codon of the VP3 polypeptide was changed into either isoleucine, leucine, or serine amino acids by PCR amplifying the insert regions and annealing with Gibson Assembly (New England Biolabs). Such vectors included the following vectors: VP1/2 (VP3 Serine mutation) (FIG. 14), VP1/2 (VP3 Leucine mutation) (FIG. 15), and VP1/2 (VP3 Isoleucine mutation) (FIG. 16).

### Construction of Vectors for Expressing Conjugating Polypeptides

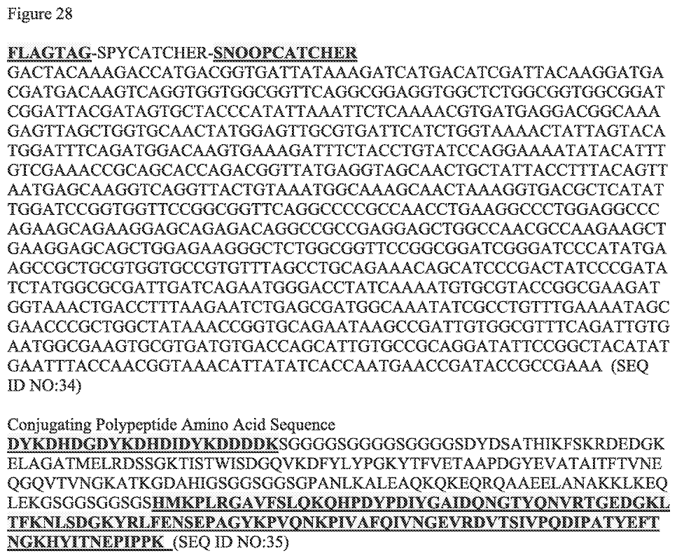

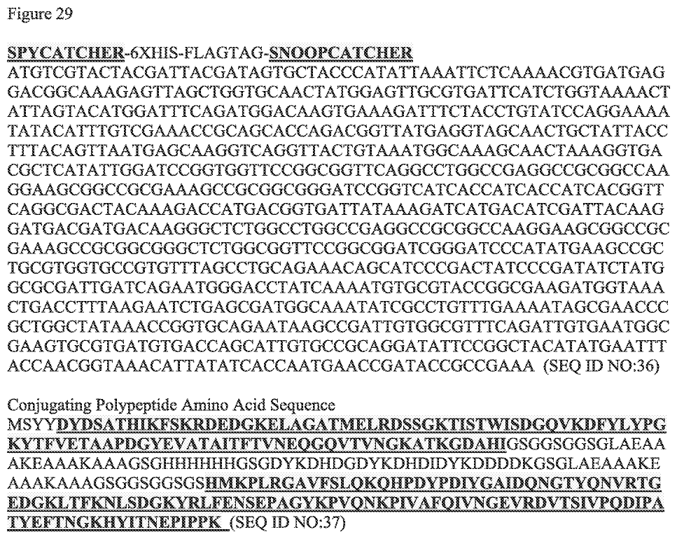

Nucleic acid encoding conjugating polypeptides was cloned into a bacterial expression vector called pET28a by PCR amplifying the insert regions and annealing with Gibson Assembly (New England Biolabs). Such vectors included: FLEX (SPYCATCHER-LONG FLEXIBLE LINKER-SNOOPCATCHER) (FIG. 27), FLAGTAG-SPYCATCHER-SNOOPCATCHER (FIG. 28), and SPYCATCHER-6XHIS-FLAGTAG-SNOOPCATCHER (FIG. 29).

### Production of AAV and Assessment of AAV Titer

In order to produce AAV vectors incorporating a heterologous amino acid segment having the ability to bind to a binding partner (e.g., a SpyTag, SnoopTag, or SpyTag002), the segment was engineered onto surface exposed regions of AAV capsid by PCR amplifying the insert regions and annealing with Gibson Assembly (New England Biolabs). The heterologous amino acid segments were inserted into position 453 or 588 of VP3 subunit of AAV vectors that does not express VP1 and VP2 polypeptides.

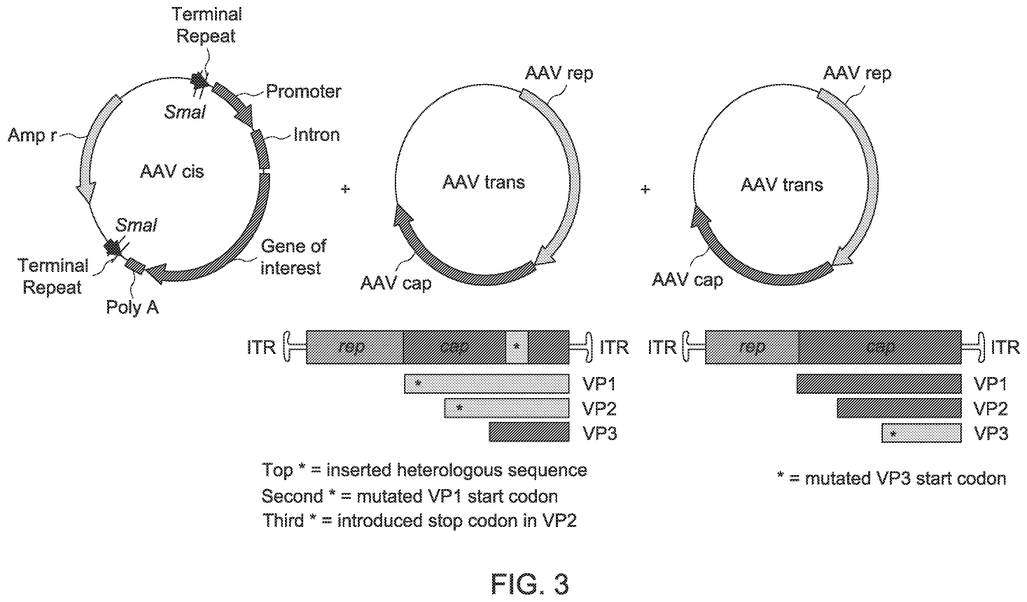

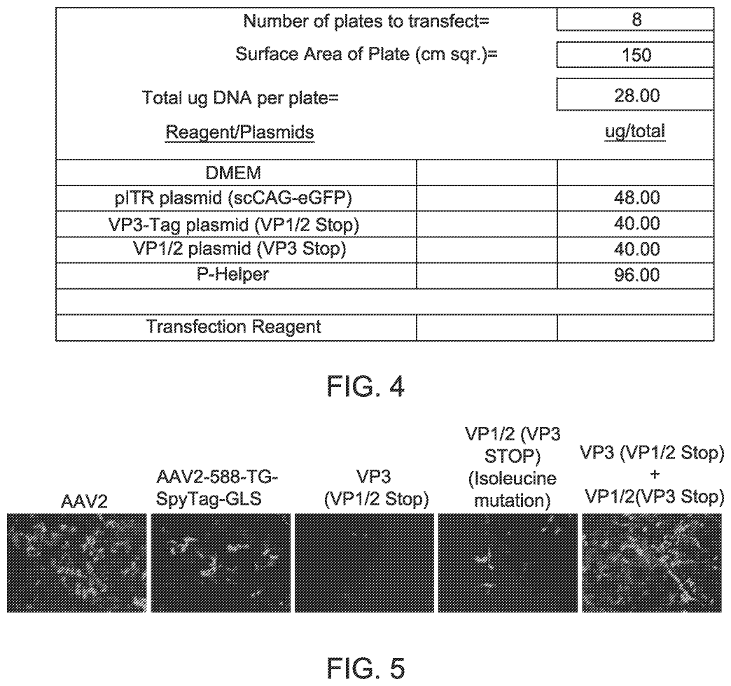

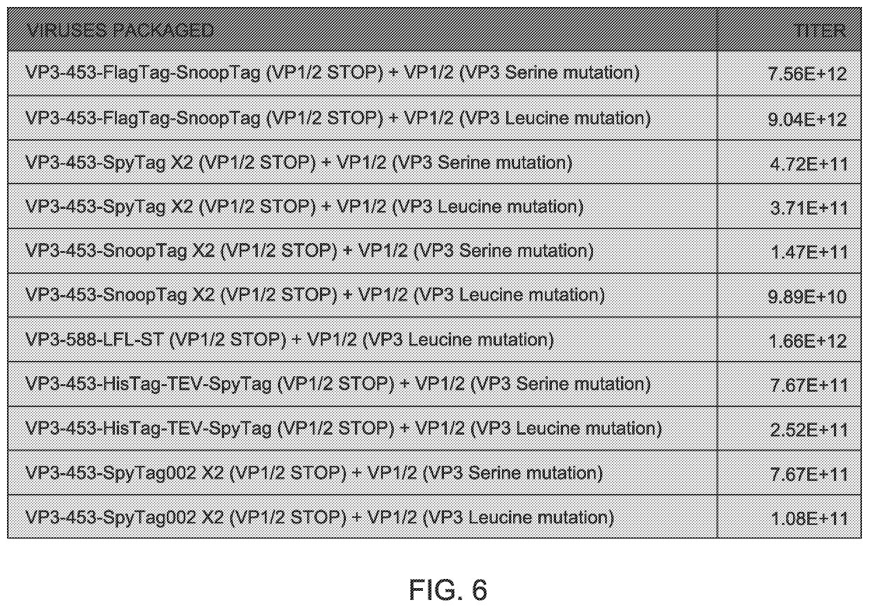

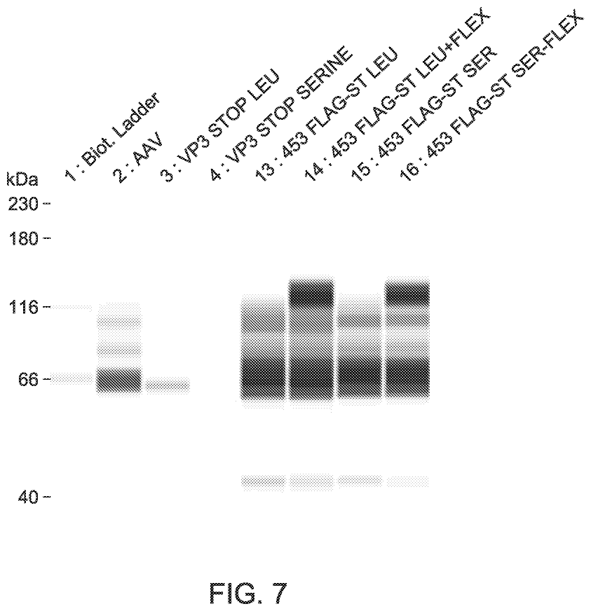

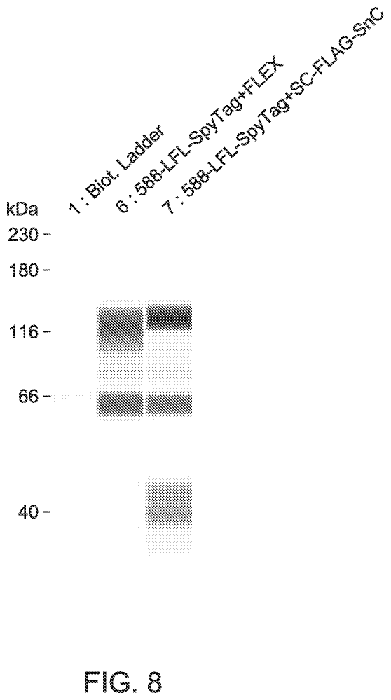

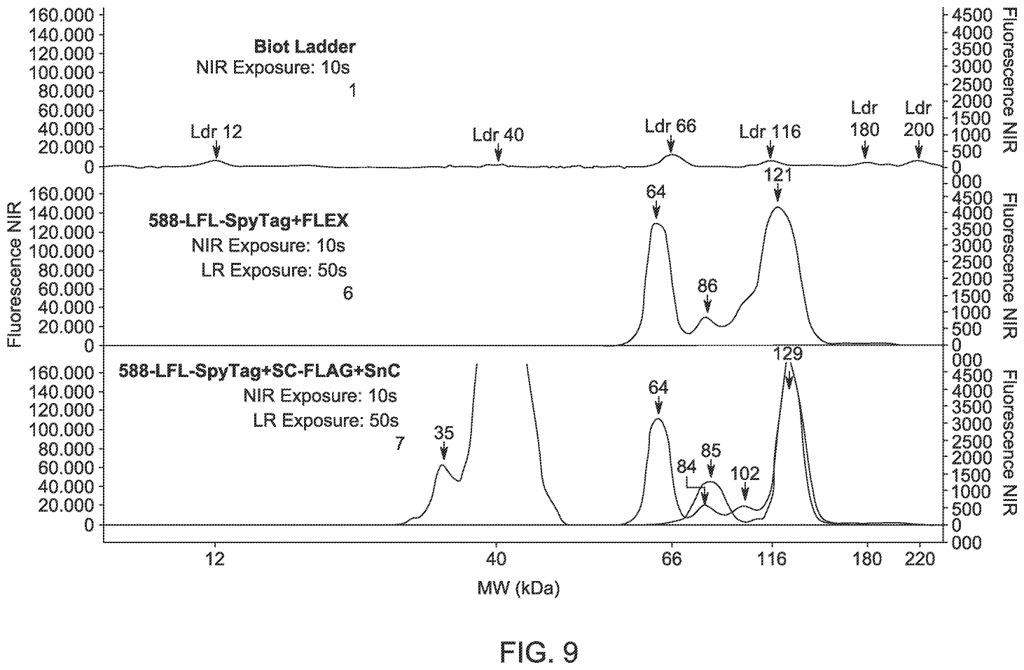

A three vector system was transfected into HEK293 cells by the plasmid co-transfection method described elsewhere (Grieger et al., Nat. Protoc., 1(3):1412-28 (2006)). The first vector was an AAV cis vector carrying a genome encoding a fluorescent protein (or any nucleic acid of interest) flanked by the viral inverted terminal repeats (ITR). The second vector was an AAV trans vector carrying a heterologous amino acid segment (e.g., a SpyTag or SnoopTag) on the VP3 subunit of the capsid. The third vector was another AAV trans vector expressing the VP1 and VP2 subunits. Recombinant AAV was purified by iodixanol gradient ultra-centrifugation followed by a buffer exchange and concentration with Amicon Ultra-15 Centrifugal Filter Units in PBS+0.001% Pluronic F-68. Titers were determined by quantitative PCR relative to a standard curve (Aurnhammer et al., Hum. Gene Ther. Methods, 23(1):18-28 (2012)). The AAV-SpyTag or AAV-Snooptag vectors were mixed with a Spycatcher/SnoopCatcher conjugating polypeptide containing a Flag Tag by incubating them at room temperature for 1 hour, followed by an overnight incubation at 4° C. Western assays were performed by Jess simple western automated immunoassay systems (Bio Techne). A mouse monoclonal antibody against VP1, VP2 and VP3 from Progen and a rabbit monoclonal antibody against Flag tag sequence from Abcam were used to demonstrate linking of the AAV-SpyTag or AAV-SnoopTag vectors to the Spycatcher/SnoopCatcher conjugating polypeptide. See, e.g., FIGS. 3-9.

### Example 2—Covalently Linking Cas9 Polypeptides to AAVs

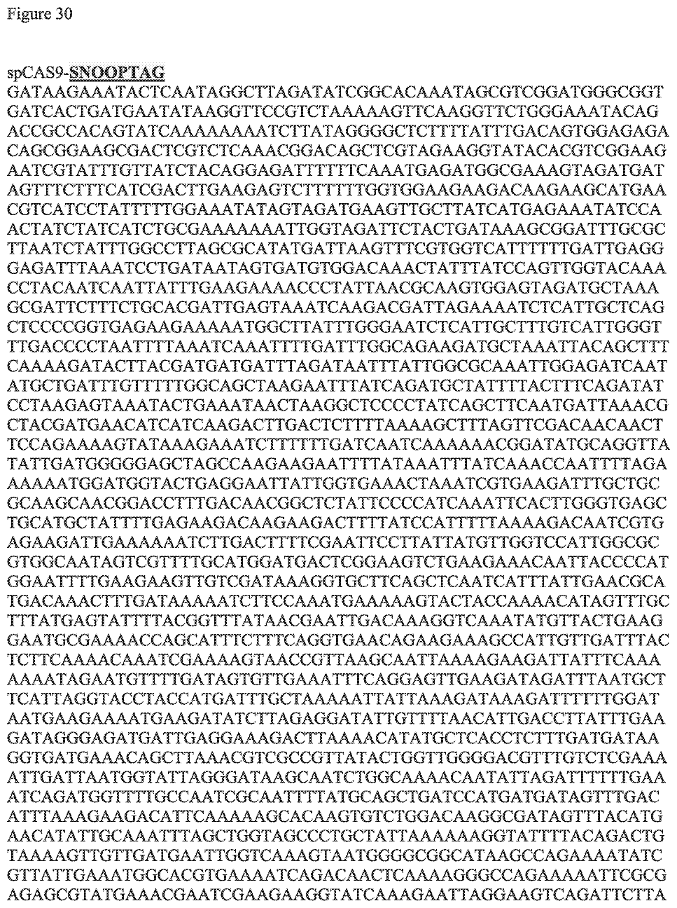

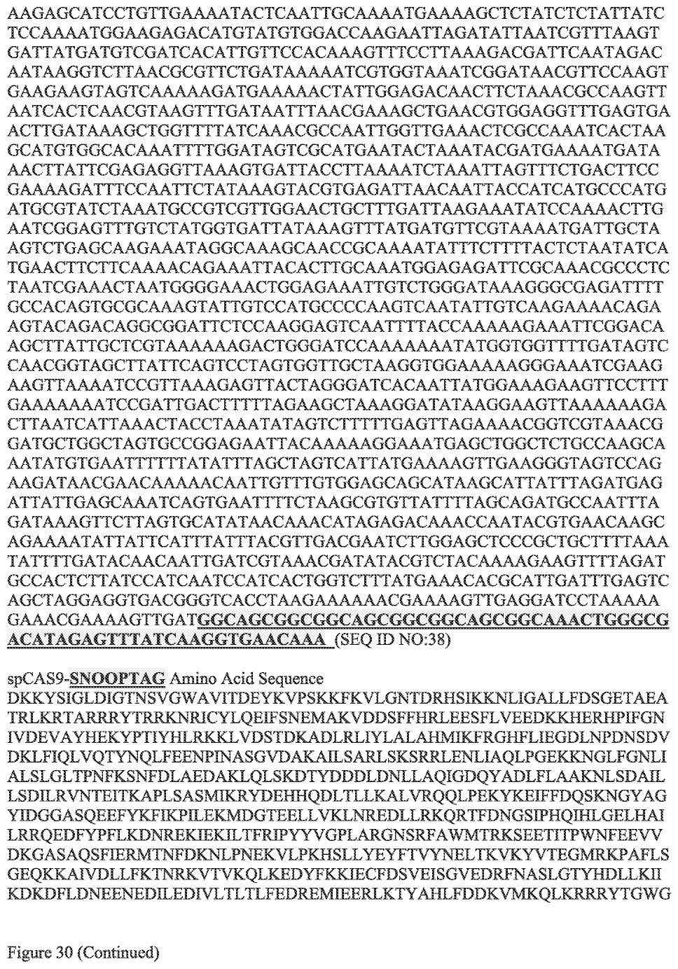

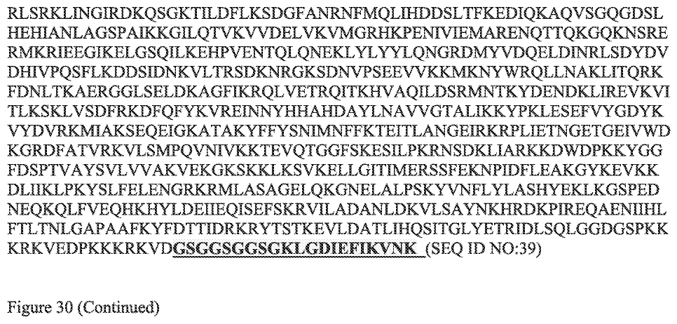

A Snooptag was fused with a Cas9 polypeptide (FIG. 30), and the Cas9-Snooptag fusion polypeptide was expressed in bacterial cells and purified. The Cas9-Snooptag fusion polypeptides were mixed with different amounts of a Spycatcher/SnoopCatcher conjugating polypeptide. In order to link the Cas9-Snooptag fusion polypeptide with the Spycatcher/SnoopCatcher conjugating polypeptide, they were incubated at room temperature for 1 hour, followed by an overnight incubation at 4° C. The mixture was run on a 6-8% Tris-Glycine gel the following day. The polypeptides were transferred to a PVDF membrane, and the membrane was blocked in 5% milk for 1 hour. The membrane was then washed 3×5 minutes in TBST and incubated in primary antibodies overnight at 4° C.: a rabbit polyclonal antibody against CRISPR-Cas9. The membrane was washed in TBST for 15 minutes followed by 4×5 minutes. Anti-rabbit secondary antibody (Li-Cor, 1:2000) was applied for 1 hour at room temperature before washing and visualization.

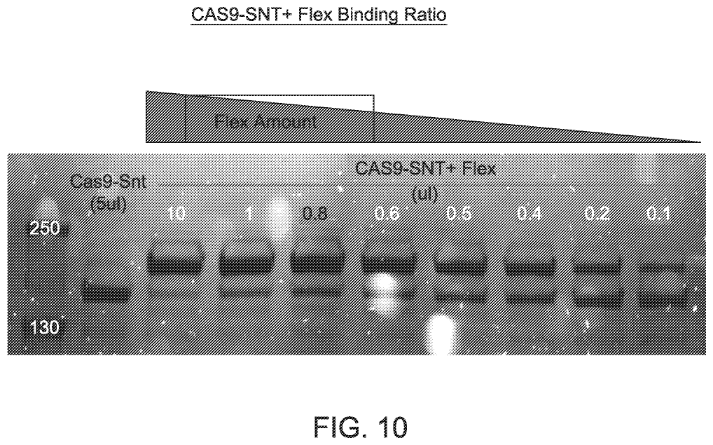

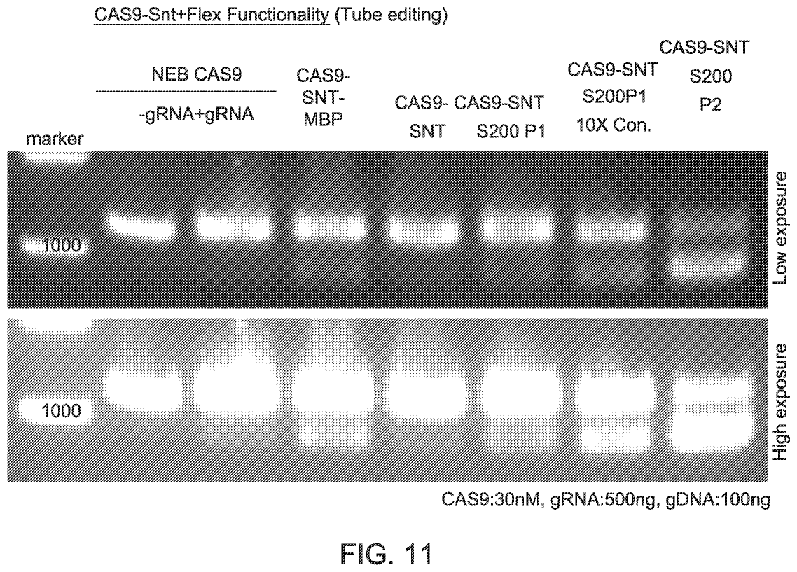

The Cas9-Snooptag fusion polypeptide was tested for its editing ability. To prepare the Cas9 RNP complexes, Cas9-Snooptag fusion polypeptide was incubated with sgRNA for 30 minutes at room temperature. gRNA was designed for targeting the human Rhodopsin gene and synthesized using GeneArt Precision gRNA Synthesis Kit (Thermo Fisher Scientific). To quantify the editing ability at desired genomic loci, T7 endonuclease I assay was performed. The samples that were PCR amplified with primers designed to amplify a ˜1 kb region containing the target site and treated with T7 endonuclease were run on an agarose gel. Results were presented in FIGS. 10-11.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An Adeno-Associated Virus (AAV) comprising a VP1 polypeptide, a VP2 polypeptide, and a VP3 polypeptide, wherein said VP3 polypeptide comprises a heterologous amino acid segment having the ability to bind to a binding partner, and wherein said VP1 polypeptide and said VP2 polypeptide lack said heterologous amino acid segment.

2. The AAV of claim 1, wherein said AAV is an AAV2.

3. The AAV of claim 1, wherein said amino acid segment comprises a spytag sequence, a snooptag sequence, a spycatcher sequence, a snoopcather sequence, or the amino acid sequence set forth in SEQ ID NO:1.

4. The AAV of claim 1, wherein said VP3 polypeptide comprises said amino acid segment at an amino acid position within three amino acid residues of an amino acid residue corresponding to position 453 or position 588 of a reference VP1 polypeptide having the amino acid sequence set forth in SEQ ID NO:2.

5. The AAV of claim 1, wherein said VP3 polypeptide comprises a protease cleavage site.

6. A composition comprising an AAV comprising a VP1 polypeptide, a VP2 polypeptide, and a VP3 polypeptide, wherein said VP3 polypeptide comprises a heterologous amino acid segment having the ability to bind to a binding partner, and wherein said VP1 polypeptide and said VP2 polypeptide lack said heterologous amino acid segment.

7. The composition of claim 6, wherein the titer of said AAV of said composition is greater than 10¹¹.

8. An isolated nucleic acid comprising a nucleic acid sequence encoding a VP1 polypeptide, a VP2 polypeptide, and a VP3 polypeptide except that said nucleic acid sequence lacks the start codon of said VP1 polypeptide, and except that said nucleic acid sequence comprises a stop codon that is (a) within the sequence encoding said VP2 polypeptide and upstream of the sequence encoding said VP3 polypeptide.

9. The isolated nucleic acid of claim 8, wherein expression of said isolated nucleic acid sequence produces said VP3 polypeptide without producing said VP1 polypeptide or said VP2 polypeptide.

10. The isolated nucleic acid of claim 8, wherein said VP3 polypeptide comprises a heterologous amino acid segment having the ability to bind to a binding partner.

11. The isolated nucleic acid of 10, wherein said amino acid segment comprises a spytag sequence, a snooptag sequence, a spycatcher sequence, a snoopcather sequence, or the amino acid sequence set forth in SEQ ID NO:1.

12. The isolated nucleic acid of claim 10, wherein said VP3 polypeptide comprises said amino acid segment at an amino acid position within three amino acid residues of an amino acid residue corresponding to position 453 or position 588 of a reference VP1 polypeptide having the amino acid sequence set forth in SEQ ID NO:2.

13. The isolated nucleic acid of claim 8, wherein said VP3 polypeptide comprises a protease cleavage site.

14. An isolated nucleic acid comprising a nucleic acid sequence encoding a VP1 polypeptide, a VP2 polypeptide, and a VP3 polypeptide except that said nucleic acid sequence lacks the start codon of said VP3 polypeptide.

15. The isolated nucleic acid of claim 14, wherein expression of said isolated nucleic acid sequence produces said VP1 polypeptide and said VP2 polypeptide without producing said VP3 polypeptide.

16. A vector system for making an AAV comprising a VP1 polypeptide, a VP2 polypeptide, and a VP3 polypeptide, wherein said VP3 polypeptide comprises a heterologous amino acid segment having the ability to bind to a binding partner, and wherein said VP1 polypeptide and said VP2 polypeptide lack said heterologous amino acid segment, wherein said vector system comprises:
(a) a first vector comprising a nucleic acid sequence encoding a VP1 polypeptide, a VP2 polypeptide, and said VP3 polypeptide comprising said heterologous amino acid segment except that said nucleic acid sequence of said first vector lacks the start codon of said VP1 polypeptide, and except that said nucleic acid sequence of said first vector comprises a stop codon that is (a) within the sequence encoding said VP2 polypeptide and upstream of the sequence encoding said VP3 polypeptide, and
(b) a second vector comprising a nucleic acid sequence encoding a VP1 polypeptide lacking said heterologous amino acid segment, a VP2 polypeptide lacking said heterologous amino acid segment, and a VP3 polypeptide except that said nucleic acid sequence of said second vector lacks the start codon of said VP3 polypeptide of said second vector.

17-21. (canceled)

22. A composition comprising (a) a first AAV comprising a VP1 polypeptide, a VP2 polypeptide, and a VP3 polypeptide comprising a heterologous amino acid segment having the ability to bind to a binding partner, wherein said VP1 and VP2 polypeptides of said first AAV lack said heterologous amino acid segment, and (b) a second AAV comprising a VP1 polypeptide, a VP2 polypeptide, and a VP3 polypeptide.

23-43. (canceled)

44. A method for producing a composition comprising a first AAV attached to a second AAV, wherein said first AAV comprises a VP1 polypeptide, a VP2 polypeptide, and a VP3 polypeptide comprising a heterologous amino acid segment having the ability to bind to a binding partner, wherein said VP1 and VP2 polypeptides of said first AAV lack said heterologous amino acid segment, and wherein said second AAV comprises a VP1 polypeptide, a VP2 polypeptide, and a VP3 polypeptide comprising said binding partner,
wherein said method comprises contacting said first AAV with said second AAV to form a first AAV-second AAV complex, thereby producing said composition.

45-46. (canceled)

47. The method of claim 44, wherein said heterologous amino acid segment comprises a spytag sequence, a snooptag sequence, a spycatcher sequence, a snoopcather sequence, or the amino acid sequence set forth in SEQ ID NO:1.

48. The method of claim 44, wherein said VP3 polypeptide of said first AAV comprises said heterologous amino acid segment at an amino acid position within three amino acid residues of an amino acid residue corresponding to position 453 or position 588 of a reference VP1 polypeptide having the amino acid sequence set forth in SEQ ID NO:2.

49. The method of claim 44, wherein said VP3 polypeptide of said first AAV comprises a protease cleavage site.

50. The method of claim 44, wherein said VP1 and VP2 polypeptides of said second AAV lack said binding partner.

51. The method of claim 44, wherein said first and second AAVs of said first AAV-second AAV complex are covalently attached to each other.

52. The method of claim 51, wherein said first AAV is covalently attached to said second AAV via at least one covalent bond between said amino acid segment of said VP3 polypeptide of said first AAV and said binding partner of said VP3 polypeptide of said second AAV.

53-65. (canceled)


SEQUENCE LISTING (74 sequences)

SEQ ID NO: 1 - protein, 13 aa, artificial
  heterologous amino acid segment of a VP3 polypeptide
DPIVMIDNDKPIT

SEQ ID NO: 2 - protein, 735 aa, artificial [sequence omitted]

SEQ ID NO: 3 - DNA, 4089 bp, artificial
  nucleic acid encoding VP1/2
acgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgtctgctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgta

SEQ ID NO: 4 - protein, 735 aa, artificial
  VP1 amino acid sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTSATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 5 - protein, 598 aa, artificial
  VP2 amino acid sequence
TAPGKKRPVEHSPVEPDSSSGTGKAGQQPARKRLNFGQTGDADSVPDPQPLGQPPAAPSGLGTNTSATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 6 - DNA, 4089 bp, artificial
  nucleic acid encoding VP1/2
acgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgcttgctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgta

SEQ ID NO: 7 - protein, 735 aa, artificial
  VP1 amino acid sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTLATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 8 - protein, 598 aa, artificial
  VP2 amino acid sequence
TAPGKKRPVEHSPVEPDSSSGTGKAGQQPARKRLNFGQTGDADSVPDPQPLGQPPAAPSGLGTNTLATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 9 - DNA, 4089 bp, artificial
  nucleic acid encoding VP1/2
acgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatcgctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgta

SEQ ID NO: 10 - protein, 735 aa, artificial
  VP1 amino acid sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTIATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 11 - protein, 598 aa, artificial
  VP2 amino acid sequence
TAPGKKRPVEHSPVEPDSSSGTGKAGQQPARKRLNFGQTGDADSVPDPQPLGQPPAAPSGLGTNTIATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 12 - DNA, 4182 bp, artificial
  nucleic acid encoding VP3-453-FlagTag-SnoopTag
acgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtaaggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggataaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaggtggcgattacaaggatgacgatgacaagggcggcagtggtggcagtggtaaactgggcgacatagagtttatcaaggtgaacaaaggtggcaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgta

SEQ ID NO: 13 - protein, 564 aa, artificial
  VP3 amino acid sequence
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGGGDYKDDDDKGGSGGSGKLGDIEFIKVNKGGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 14 - DNA, 4197 bp, artificial
  nucleic acid encoding VP3-588-Long Flexible Linker-SPYTAG
acgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtaaggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggataaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagatcaggtggtagtggcggttcaggcggaggtggctctggcggttctggcggatcgggcaacggtggcagtgcccacatcgtgatggtggacgcctacaagccgacgaagggttctagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgta

SEQ ID NO: 15 - protein, 569 aa, artificial
  VP3 amino acid sequence
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRSGGSGGSGGGGSGGSGGSGNGGSAHIVMVDAYKPTKGSRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 16 - DNA, 4188 bp, artificial
  nucleic acid encoding VP3-453-SPYTAG-SPYTAG
acgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtaaggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggataaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaggtggcgcccacatcgtgatggtggacgcctacaagccgacgaagggcagtggtgcccacatcgtgatggtggacgcctacaagccgacgaagggtggcaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgta

SEQ ID NO: 17 - protein, 566 aa, artificial
  VP3 amino acid sequence
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGGGAHIVMVDAYKPTKGSGAHIVMVDAYKPTKGGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 18 - DNA, 4182 bp, artificial
  nucleic acid encoding VP3-453-SNOOPTAG-SNOOPTAG
acgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtaaggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggataaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaggtggcaaactgggcgacatagagtttatcaaggtgaacaaaggcagtggtaaactgggcgacatagagtttatcaaggtgaacaaaggtggcaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgta

SEQ ID NO: 19 - protein, 564 aa, artificial
  VP3 amino acid sequence
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGGGKLGDIEFIKVNKGSGKLGDIEFIKVNKGGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 20 - DNA, 4188 bp, artificial
  nucleic acid encoding VP3-453-HisTag-TEV-SpyTag
acgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtaaggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggataaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaggtggccatcaccatcaccatcacgagaacctctatttccagggaggcagtggtgcccacatcgtgatggtggacgcctacaagccgacgaagggtggcaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgta

SEQ ID NO: 21 - protein, 566 aa, artificial
  VP3 amino acid sequence
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGGGHHHHHHENLYFQGGSGAHIVMVDAYKPTKGGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 22 - DNA, 4194 bp, artificial
  nucleic acid encoding VP3-453-SpyTag002 X2
acgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtaaggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggataaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaggtggcgtgcctactatcgtgatggtggacgcctacaagcgttacaagggcagtggtgtgcctactatcgtgatggtggacgcctacaagcgttacaagggtggcaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgta

SEQ ID NO: 23 - protein, 568 aa, artificial
  VP3 amino acid sequence
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGGGVPTIVMVDAYKRYKGSGVPTIVMVDAYKRYKGGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 24 - DNA, 4176 bp, artificial
  nucleic acid encoding VP3-588-SnoopTag X2
acgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtaaggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggataaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggtggcaaactgggcgacatagagtttatcaaggtgaacaaaggcagtggtaaactgggcgacatagagtttatcaaggtgaacaaaggtggcagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgta

SEQ ID NO: 25 - protein, 562 aa, artificial
  VP3 amino acid sequence
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGGKLGDIEFIKVNKGSGKLGDIEFIKVNKGGRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 26 - DNA, 4203 bp, artificial
  nucleic acid encoding VP3-453-LFL-SpyTag
acgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtaaggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggataaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggatcaggtggtagtggcggttcaggcggaggtggctctggcggttctggcggatcgggcaacggtggcagtgcccacatcgtgatggtggacgcctacaagccgacgaagggtggcaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgta

SEQ ID NO: 27 - protein, 571 aa, artificial
  VP3 amino acid sequence
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGSGGSGGSGGGGSGGSGGSGNGGSAHIVMVDAYKPTKGGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 28 - DNA, 4185 bp, artificial
  nucleic acid encoding VP3-453-FlagTag-SpyTag
acgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtaaggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggataaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaggtggcgattacaaggatgacgatgacaagggcggcagtggtggcagtggtgcccacatcgtgatggtggacgcctacaagccgacgaagggtggcaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgta

SEQ ID NO: 29 - protein, 565 aa, artificial
  VP3 amino acid sequence
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGGGDYKDDDDKGGSGGSGAHIVMVDAYKPTKGGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 30 - DNA, 4194 bp, artificial
  nucleic acid encoding VP3-588-LFL-SnoopTag
acgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtaaggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggataaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagatcaggtggtagtggcggttcaggcggaggtggctctggcggttctggcggatcgggcaacggtggcagtaaactgggcgacatagagtttatcaaggtgaacaaaggttctagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgta

SEQ ID NO: 31 - protein, 568 aa, artificial
  VP3 amino acid sequence
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRSGGSGGSGGGGSGGSGGSGNGGSKLGDIEFIKVNKGSRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 32 - DNA, 783 bp, artificial
  nucleic acid encoding FLEX
gattacgatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatattggatccggtggttccggcggttcaggccccgccaacctgaaggccctggaggcccagaagcagaaggagcagagacaggccgccgaggagctggccaacgccaagaagctgaaggagcagctggagaagggctctggcggttccggcggatcgggatcccatatgaagccgctgcgtggtgccgtgtttagcctgcagaaacagcatcccgactatcccgatatctatggcgcgattgatcagaatgggacctatcaaaatgtgcgtaccggcgaagatggtaaactgacctttaagaatctgagcgatggcaaatatcgcctgtttgaaaatagcgaacccgctggctataaaccggtgcagaataagccgattgtggcgtttcagattgtgaatggcgaagtgcgtgatgtgaccagcattgtgccgcaggatattccggctacatatgaatttaccaacggtaaacattatatcaccaatgaaccgataccgccgaaa

SEQ ID NO: 33 - protein, 261 aa, artificial
  amino acid sequence of FLEX
DYDSATHIKFSKRDEDGKELAGATMELRDSSGKTISTWISDGQVKDFYLYPGKYTFVETAAPDGYEVATAITFTVNEQGQVTVNGKATKGDAHIGSGGSGGSGPANLKALEAQKQKEQRQAAEELANAKKLKEQLEKGSGGSGGSGSHMKPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 34 - DNA, 897 bp, artificial
  nucleic acid encoding a FLAGTAG-SPYCATCHER- SNOOPCATCHER conjugating polypeptide
gactacaaagaccatgacggtgattataaagatcatgacatcgattacaaggatgacgatgacaagtcaggtggtggcggttcaggcggaggtggctctggcggtggcggatcggattacgatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatattggatccggtggttccggcggttcaggccccgccaacctgaaggccctggaggcccagaagcagaaggagcagagacaggccgccgaggagctggccaacgccaagaagctgaaggagcagctggagaagggctctggcggttccggcggatcgggatcccatatgaagccgctgcgtggtgccgtgtttagcctgcagaaacagcatcccgactatcccgatatctatggcgcgattgatcagaatgggacctatcaaaatgtgcgtaccggcgaagatggtaaactgacctttaagaatctgagcgatggcaaatatcgcctgtttgaaaatagcgaacccgctggctataaaccggtgcagaataagccgattgtggcgtttcagattgtgaatggcgaagtgcgtgatgtgaccagcattgtgccgcaggatattccggctacatatgaatttaccaacggtaaacattatatcaccaatgaaccgataccgccgaaa

SEQ ID NO: 35 - protein, 299 aa, artificial
  amino acid sequence of a FLAGTAG-SPYCATCHER- SNOOPCATCHER conjugating polypeptide
DYKDHDGDYKDHDIDYKDDDDKSGGGGSGGGGSGGGGSDYDSATHIKFSKRDEDGKELAGATMELRDSSGKTISTWISDGQVKDFYLYPGKYTFVETAAPDGYEVATAITFTVNEQGQVTVNGKATKGDAHIGSGGSGGSGPANLKALEAQKQKEQRQAAEELANAKKLKEQLEKGSGGSGGSGSHMKPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 36 - DNA, 894 bp, artificial
  nucleic acid encoding a SPYCATCHER-6XHIS- FLAGTAG-SNOOPCATCHER conjugating polypeptide
atgtcgtactacgattacgatagtgctacccatattaaattctcaaaacgtgatgaggacggcaaagagttagctggtgcaactatggagttgcgtgattcatctggtaaaactattagtacatggatttcagatggacaagtgaaagatttctacctgtatccaggaaaatatacatttgtcgaaaccgcagcaccagacggttatgaggtagcaactgctattacctttacagttaatgagcaaggtcaggttactgtaaatggcaaagcaactaaaggtgacgctcatattggatccggtggttccggcggttcaggcctggccgaggccgcggccaaggaagcggccgcgaaagccgcggcgggatccggtcatcaccatcaccatcacggttcaggcgactacaaagaccatgacggtgattataaagatcatgacatcgattacaaggatgacgatgacaagggctctggcctggccgaggccgcggccaaggaagcggccgcgaaagccgcggcgggctctggcggttccggcggatcgggatcccatatgaagccgctgcgtggtgccgtgtttagcctgcagaaacagcatcccgactatcccgatatctatggcgcgattgatcagaatgggacctatcaaaatgtgcgtaccggcgaagatggtaaactgacctttaagaatctgagcgatggcaaatatcgcctgtttgaaaatagcgaacccgctggctataaaccggtgcagaataagccgattgtggcgtttcagattgtgaatggcgaagtgcgtgatgtgaccagcattgtgccgcaggatattccggctacatatgaatttaccaacggtaaacattatatcaccaatgaaccgataccgccgaaa

SEQ ID NO: 37 - protein, 298 aa, artificial
  amino acid sequence of a SPYCATCHER-6XHIS- FLAGTAG-SNOOPCATCHER conjugating polypeptide
MSYYDYDSATHIKFSKRDEDGKELAGATMELRDSSGKTISTWISDGQVKDFYLYPGKYTFVETAAPDGYEVATAITFTVNEQGQVTVNGKATKGDAHIGSGGSGGSGLAEAAAKEAAAKAAAGSGHHHHHHGSGDYKDHDGDYKDHDIDYKDDDDKGSGLAEAAAKEAAAKAAAGSGGSGGSGSHMKPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 38 - DNA, 4221 bp, artificial
  nucleic acid encoding a spCAS9-SNOOPTAG polypeptide
gataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgacgggtcacctaagaaaaaacgaaaagttgaggatcctaaaaagaaacgaaaagttgatggcagcggcggcagcggcggcagcggcaaactgggcgacatagagtttatcaaggtgaacaaa

SEQ ID NO: 39 - protein, 1407 aa, artificial
  amino acid sequence of a spCAS9-SNOOPTAG polypeptide
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGSPKKKRKVEDPKKKRKVDGSGGSGGSGKLGDIEFIKVNK

SEQ ID NO: 40 - protein, 13 aa, artificial
  variant of SEQ ID NO1
APIVMIDNDKPIT

SEQ ID NO: 41 - protein, 13 aa, artificial
  variant of SEQ ID NO1
DHIVMIDNDKPIT

SEQ ID NO: 42 - protein, 13 aa, artificial
  variant of SEQ ID NO1
DPIVMVDNDKPIT

SEQ ID NO: 43 - protein, 13 aa, artificial
  variant of SEQ ID NO1
DPIVMIDADKPIT

SEQ ID NO: 44 - protein, 13 aa, artificial
  variant of SEQ ID NO1
DPIVMIDNYKPIT

SEQ ID NO: 45 - protein, 13 aa, artificial
  variant of SEQ ID NO1
DPIVMIDNDKPYT

SEQ ID NO: 46 - protein, 13 aa, artificial
  variant of SEQ ID NO1
DPIVMIDNDKPIT

SEQ ID NO: 47 - protein, 17 aa, artificial
  variant of SEQ ID NO1
GSDPIVMIDNDKPITGS

SEQ ID NO: 48 - protein, 12 aa, artificial
  variant of SEQ ID NO1
PIVMIDNDKPIT

SEQ ID NO: 49 - protein, 11 aa, artificial
  variant of SEQ ID NO1
PIVMIDNDKPI

SEQ ID NO: 50 - protein, 7 aa, artificial
  TEV protease cleavage site
ENLYFQG

SEQ ID NO: 51 - protein, 7 aa, artificial
  PreScission protease cleavage site
  MISC_FEATURE (6)..(6): X is Q or G
LEVLFXP

SEQ ID NO: 52 - protein, 6 aa, artificial
  Thrombin protease cleavage site
LVPRGS

SEQ ID NO: 53 - protein, 4 aa, artificial
  Factor Xa protease cleavage site
  MISC_FEATURE (2)..(2): X is E or P
IXGR

SEQ ID NO: 54 - protein, 5 aa, artificial
  Enterokinase protease cleavage site
DDDDK

SEQ ID NO: 55 - protein, 8 aa, artificial
  CleanCut protease cleavage site
LVPWELQE

SEQ ID NO: 56 - protein, 83 aa, artificial
  binding partner sequences having the ability to form a covalent bond with SEQ ID NO1 (or a variant thereof)
  misc_feature (6)..(6): Xaa can be any naturally occurring amino acid
  misc_feature (16)..(16): Xaa can be any naturally occurring amino acid
  misc_feature (22)..(22): Xaa can be any naturally occurring amino acid
  misc_feature (28)..(28): Xaa can be any naturally occurring amino acid
  misc_feature (31)..(31): Xaa can be any naturally occurring amino acid
  misc_feature (37)..(37): Xaa can be any naturally occurring amino acid
  misc_feature (41)..(41): Xaa can be any naturally occurring amino acid
  misc_feature (51)..(51): Xaa can be any naturally occurring amino acid
  misc_feature (66)..(66): Xaa can be any naturally occurring amino acid
  misc_feature (76)..(76): Xaa can be any naturally occurring amino acid
  misc_feature (82)..(82): Xaa can be any naturally occurring amino acid
DSTTHXKFSKRDINGXELAGAXIELRNXSGXTIQSWXSDGXVKDFYLMPGXYQFVETAAPEGYELXAPITFTIDEXGQIWVXS

SEQ ID NO: 57 - protein, 83 aa, artificial
  binding partner sequences having the ability to form a covalent bond with SEQ ID NO1 (or a variant thereof)
  MISC_FEATURE (6)..(6): X at residue 6 = valine, alanine, isoleucine, or leucine
  MISC_FEATURE (16)..(16): X at residue 16 = lysine, arginine, histidine, or serine
  MISC_FEATURE (22)..(22): X at residue 22 = methionine, leucine, phenylalanine, or tryptophan
  MISC_FEATURE (28)..(28): X at residue 28 = leucine, methionine, isoleucine, or tyrosine
  MISC_FEATURE (31)..(31): X at residue 31 = glutamine, serine, threonine, or asparagine
  MISC_FEATURE (37)..(37): X at residue 37 = isoleucine, leucine, methionine, or phenylalanine
  MISC_FEATURE (41)..(41): X at residue 41 = threonine, serine, valine, or asparagine
  MISC_FEATURE (44)..(44): X at residue 44 is D or V
  MISC_FEATURE (51)..(51): X at residue 51 = threonine, serine, valine, or asparagine
  MISC_FEATURE (66)..(66): X at residue 66 = alanine, isoleucine, leucine, or phenylalanine
  MISC_FEATURE (76)..(76): X at residue 76 = lysine, arginine, histidine, or serine
  MISC_FEATURE (82)..(82): X at residue 82 = aspartic acid, glutamic acid, or lysine
DSTTHXKFSKRDINGXELAGAXIELRNXSGXTIQSWXSDGXVKXFYLMPGXYQFVETAAPEGYELXAPITFTIDEXGQIWVXS

SEQ ID NO: 58 - protein, 123 aa, artificial
  binding partner sequences having the ability to form a covalent bond with SEQ ID NO1 (or a variant thereof)
  misc_feature (4)..(4): Xaa can be any naturally occurring amino acid
  misc_feature (9)..(10): Xaa can be any naturally occurring amino acid
  misc_feature (12)..(13): Xaa can be any naturally occurring amino acid
  misc_feature (15)..(15): Xaa can be any naturally occurring amino acid
  misc_feature (19)..(19): Xaa can be any naturally occurring amino acid
  misc_feature (27)..(27): Xaa can be any naturally occurring amino acid
  misc_feature (37)..(37): Xaa can be any naturally occurring amino acid
  misc_feature (43)..(43): Xaa can be any naturally occurring amino acid
  misc_feature (49)..(49): Xaa can be any naturally occurring amino acid
  misc_feature (52)..(52): Xaa can be any naturally occurring amino acid
  misc_feature (58)..(58): Xaa can be any naturally occurring amino acid
  misc_feature (62)..(62): Xaa can be any naturally occurring amino acid
  MISC_FEATURE (65)..(65): X at residue 65 is D or V
  misc_feature (72)..(72): Xaa can be any naturally occurring amino acid
  misc_feature (87)..(87): Xaa can be any naturally occurring amino acid
  misc_feature (97)..(97): Xaa can be any naturally occurring amino acid
  misc_feature (103)..(103): Xaa can be any naturally occurring amino acid
  misc_feature (105)..(105): Xaa can be any naturally occurring amino acid
  misc_feature (113)..(113): Xaa can be any naturally occurring amino acid
IDTXSGLSXXTXXSXNTTXEEDSTTHXKFSKRDINGXELAGAXIELRNXSGXTIQSWXSDGXVKXFYLMPGXYQFVETAAPEGYELXAPITFTIDEXGQIWVXSXLIVGDDPXVMIDNDKPIT

SEQ ID NO: 59 - protein, 123 aa, artificial; identical to SEQ ID NO: 58
  binding partner sequences having the ability to form a covalent bond with SEQ ID NO1 (or a variant thereof)
  MISC_FEATURE (4)..(4): X at residue 4 = methionine, isoleucine, leucine, or phenylalanine
  MISC_FEATURE (9)..(9): X at residue 9 = glycine, proline, cysteine, or serine
  MISC_FEATURE (10)..(10): X at residue 10 = glutamic acid, aspartic acid, arginine, or lysine
  MISC_FEATURE (12)..(12): X at residue 12 = glycine, proline, cysteine, or serine
  MISC_FEATURE (13)..(13): X at residue 13 = glutamine, serine, threonine, or asparagine
  MISC_FEATURE (15)..(15): X at residue 15 = glycine, proline, cysteine, or serine
  MISC_FEATURE (19)..(19): X at residue 19 = isoleucine, leucine, methionine, or phenylalanine
  MISC_FEATURE (27)..(27): X at residue 27 = valine, alanine, isoleucine, or leucine
  MISC_FEATURE (37)..(37): X at residue 37 = lysine, arginine, histidine, or serine
  MISC_FEATURE (43)..(43): X at residue 43 = methionine, leucine, phenylalanine, or tryptophan
  MISC_FEATURE (49)..(49): X at residue 49 = leucine, methionine, isoleucine, or tyrosine
  MISC_FEATURE (52)..(52): X at residue 52 = glutamine, serine, threonine, or asparagine
  MISC_FEATURE (58)..(58): X at residue 58 = isoleucine, leucine, methionine, or phenylalanine
  MISC_FEATURE (62)..(62): X at residue 62 = threonine, serine, valine, or asparagine
  MISC_FEATURE (65)..(65): X at residue 65 is D or V
  MISC_FEATURE (65)..(65): X at residue 65 is D or V
  MISC_FEATURE (72)..(72): X at residue 72 = threonine, serine, valine, or asparagine
  MISC_FEATURE (87)..(87): X at residue 87 = alanine, isoleucine, leucine, or phenylalanine
  MISC_FEATURE (97)..(97): X at residue 97 = lysine, arginine, histidine, or serine
  MISC_FEATURE (103)..(103): X at residue 103 = aspartic acid, glutamic acid, or lysine
  MISC_FEATURE (105)..(105): X at residue 105 = threonine, serine, valine, or asparagine
  MISC_FEATURE (113)..(113): X at residue 113 = isoleucine, leucine, methionine, or phenylalanine;

SEQ ID NO: 60 - protein, 83 aa, artificial
  binding partner sequences having the ability to form a covalent bond with SEQ ID NO1 (or a variant thereof)
  MISC_FEATURE (44)..(44): X is D or V
DSTTHVKFSKRDINGKELAGAMIELRNLSGQTIQSWVSDGTVKXFYLMPGTYQFVETAAPEGYELAAPITFTIDEKGQIWVDS

SEQ ID NO: 61 - protein, 123 aa, artificial
  binding partner sequences having the ability to form a covalent bond with SEQ ID NO1 (or a variant thereof)
  MISC_FEATURE (65)..(65): X is D or V
IDTMSGLSGETGQSGNTTIEEDSTTHVKFSKRDINGKELAGAMIELRNLSGQTIQSWVSDGTVKXFYLMPGTYQFVETAAPEGYELAAPITFTIDEKGQIWVDSTLIVGDDPIVMIDNDKPIT

SEQ ID NO: 62 - protein, 111 aa, artificial
  binding partner sequences having the ability to form a covalent bond with SEQ ID NO1 (or a variant thereof)
  MISC_FEATURE (64)..(64): X is D or V
DTMSGLSGETGQSGNTTIEEDSTTHVKFSKRDINGKELAGAMIELRNLSGQTIQSWVSDGTVKXFYLMPGTYQFVETAAPEGYELAAPITFTIDEKGQIWVDSTLIVGDDP

SEQ ID NO: 63 - protein, 103 aa, artificial
  binding partner sequences having the ability to form a covalent bond with SEQ ID NO1 (or a variant thereof)
  MISC_FEATURE (64)..(64): X is D or V
DTMSGLSGETGQSGNTTIEEDSTTHVKFSKRDINGKELAGAMIELRNLSGQTIQSWVSDGTVKXFYLMPGTYQFVETAAPEGYELAAPITFTIDEKGQIWVDS

SEQ ID NO: 64 - protein, 102 aa, artificial
  binding partner sequences having the ability to form a covalent bond with SEQ ID NO1 (or a variant thereof)
  MISC_FEATURE (44)..(44): X is D or V
DSTTHVKFSKRDINGKELAGAMIELRNLSGQTIQSWVSDGTVKXFYLMPGTYQFVETAAPEGYELAAPITFTIDEKGQIWVDSTLIVGDDPIVMIDNDKPIT

SEQ ID NO: 65 - protein, 122 aa, artificial
  binding partner sequences having the ability to form a covalent bond with SEQ ID NO1 (or a variant thereof)
  MISC_FEATURE (64)..(64): X is D or V
DTMSGLSGETGQSGNTTIEEDSTTHVKFSKRDINGKELAGAMIELRNLSGQTIQSWVSDGTVKXFYLMPGTYQFVETAAPEGYELAAPITFTIDEKGQIWVDSTLIVGDDPIVMIDNDKPIT

SEQ ID NO: 66 - protein, 16 aa, artificial
  polypeptide linker
SGGGGSGGGGSGGGGS

SEQ ID NO: 67 - protein, 15 aa, artificial
  polypeptide linker
LAEAAAKEAAAKAAA

SEQ ID NO: 68 - protein, 54 aa, artificial
  polypeptide linker
GSGGGSGGGSPANLKALEAQKQKEQRQAAEELANAKKLKEQLEKGSGGGSGGGS

SEQ ID NO: 69 - protein, 13 aa, artificial
  heterologous amino acid segment
AHIVMVDAYKPTK

SEQ ID NO: 70 - protein, 92 aa, artificial
  heterologous amino acid segment binding partner
DSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 71 - protein, 12 aa, artificial
  heterologous amino acid segment
KLGDIEFIKVNK

SEQ ID NO: 72 - protein, 116 aa, artificial
  heterologous amino acid segment binding partner
GSHMKPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 73 - protein, 14 aa, artificial
  heterologous amino acid segment
VPTIVMVDAYKRYK

SEQ ID NO: 74 - protein, 120 aa, artificial
  heterologous amino acid segment binding partner
AMVTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHTGSSGS